(19) United States
(12) Patent Application Publication
(10) Pub. No.: US 2025/0139767 A1
(43) Pub. Date: May 1, 2025

(54) MACHINE LEARNING FOR PREDICTING CANCER GENOTYPE AND TREATMENT RESPONSE USING DIGITAL HISTOPATHOLOGY IMAGES

(71) Applicant: Janssen Research & Development, LLC, Raritan, NJ (US)

(72) Inventors: Albert Juan Ramon, Raritan, NJ (US); Kristopher Standish, Raritan, NJ (US); Chaitanya Parmar, Raritan, NJ (US); Stephen Yip, Raritan, NJ (US); Joel Greshock, Raritan, NJ (US)

(73) Assignee: Janssen Research & Development, LLC, Raritan, NJ (US)

(21) Appl. No.: 18/693,516

(22) PCT Filed: Sep. 20, 2022
(86) PCT No.: PCT/IB2022/058892

§ 371 Date: Mar. 19, 2024

Related U.S. Application Data
(60) Provisional application No. 63/301,023, filed on Jan. 19, 2022.
(60) Provisional application No. 63/246,178, filed on Sep. 20, 2021.

(51) Int. Cl. G06T 7/00; G06V 10/764; G06V 10/77; G06V 10/774; G06V 10/82; G06V 20/69; G16H 50/20

(52) U.S. Cl. CPC G06T 7/0012; G06V 10/764; G06V 10/7715; G06V 10/774; G06V 10/82; G06V 20/698; G16H 50/20; G06T 2207/20081; G06T 2207/20084; G06T 2207/30024; G06T 2207/30096; G06V 2201/03

(57) ABSTRACT

Computerized systems and methods for digital histopathology analysis are disclosed. In one embodiment, a series of deep learning networks are used that train, in succession, on datasets of successively increasing relevance. In some examples, learned parameters from at least a portion of one deep learning network are transferred to a next deep learning network in a succession of deep learning networks. In some examples, at least one of the deep learning networks includes a self-supervised learning network. In some examples, at least one of the deep learning networks includes an attention-based learning network. These and other examples and details are disclosed herein in various contexts including, for example evaluating genotypes of cancer tissue (e.g., bladder, prostate, or lung cancer) using histopathology images. In some examples, the context is to assist in predicting presence or absence of certain cancer genotypes and/or predicting patient responses to a new treatment.

### CROSS-REFERENCE TO RELATED APPLICATIONS

This application claims the benefit of U.S. Provisional Application No. 63/246,178 filed on Sep. 20, 2021 and U.S. Provisional Application No. 63/301,023 filed on Jan. 19, 2022. The contents of these applications are herein incorporated by reference.

### BACKGROUND

This disclosure relates generally to technology for computerized prediction of cancer genotypes and treatment response using tissue images.

### SUMMARY

Recruiting patients for clinical trials for drugs targeting patients with specific cancer mutations is slow and costly. One example is in the context of treating bladder cancer with erdafitinib; another example is in the context of treating prostate cancer treated with niraparib; and another example is in the context of treating lung cancer treated with amivantamab. Once a cancer patient is diagnosed, the patient could be referred to available clinical trial sites. Patient biopsies are taken as part of diagnoses and undergo molecular screening to test for the specific gene mutations/alterations targeted by the clinical trial drug. However, such screening can be expensive and obtaining the results can take an undesirably long term. Furthermore, the tests require tumor tissue that cannot be replaced. Some patients may decide to forego molecular screening (or undergo a different molecular test) if they perceive their likelihood of having one of the qualifying mutations to be low. Moreover, the faster that appropriate candidates for clinical trials can be identified, the faster the efficacy of potentially life-saving new treatments can be determined. For these and other reasons, easier and more effective prediction of specific cancer genotypes, especially in the context of screening candidates clinical trial participation, is needed.

Machine learning systems have been used to analyze digital histopathology images. However, these systems generally depend on large sets of relevant labelled training data to train computerized systems to assist in assessing, diagnosing, or making predictions based on histopathology image data. But in the context of clinical trials for new and potentially promising treatments, large sets of labeled histopathology image data corresponding to patients who have undergone such treatments and have known outcomes are generally not available.

Embodiments of the present disclosure include a computerized pipeline system and method that can effectively learn to make predictions in situations where the histopathology image sets of highest relevance are necessarily relatively small. For example, a cohort of interest might include participants (or candidates for participation) in a clinical trial for a new therapeutic treatment for a particular disease. The new treatment might include, for example, administering new drug, a new combination of drugs, and/or a using new protocol for treating the relevant disease. For example, a small dataset might include images from only 100-200 patients or even fewer than 100 patients.

The challenge posed by such small histopathology image datasets is that it is generally too difficult or not possible to effectively train a deep learning network from scratch to make sufficiently accurate predictions using such a small amount of training data. However, it may be possible to use such a small dataset to fine-tune/train a deep learning network that has a feature extraction portion (and/or other portions) that have been pre-trained on other datasets, preferably, a series of datasets starting with large, less relevant datasets and continuing with increasingly relevant (and often smaller) datasets.

For many diseases, such as cancer, there are large publicly available datasets (e.g., The Cancer Genome Atlas—“TCGA”—data) that might correspond to histopathology images (e.g., whole slide images) from more than 20,000 or even more than 30,000 individuals. Although such datasets will often identify the type of disease (e.g., type of cancer), they will not necessarily also identify other specific information that might be needed for using fully supervised learning for predicting specific tumor genotypes and/or predicting responses to various treatments. Thus, such datasets might be “unlabeled” and/or less relevant to a cohort of interest than other, smaller datasets in the sense that specific known information regarding treatment results or even identifying specific tumor genotypes within a specific type of cancer (e.g., particular genotypes for specific mutations relevant to bladder, prostate, lung, etc.), might not be available.

However, such large, unlabeled datasets can be effectively used as part of a pipeline system for developing a trained network using successively more relevant (and potentially significantly smaller) datasets for developing a trained deep learning network for analyzing data corresponding to a cohort of interest such as candidates or participants in a clinical trial for a particular new treatment.

In one embodiment of a pipeline system in accordance with the present disclosure, a first deep learning network is used to perform self-supervised learning (which does not require labeled training data) on a large histopathology image dataset to begin training a feature extraction portion (and/or other portions) of a deep learning network. In one example, a contrastive learning network is used. However, in other implementations consistent with the principles of this disclosure, a first deep learning network in the pipeline might be configured to implement other types of self-supervised learning. In other alternatives, it might be configured to implement a learning type other than self-supervised learning.

In selected embodiments, subsequent datasets of increasing relevance to a cohort of interest can be used to train, in succession, additional deep learning networks in the pipeline. These additional networks can be configured to use supervised learning, which, in some examples, includes attention-based multiple instance learning. Learned weights can be transferred from a feature extraction portion (and/or additional portions) of one network in the pipeline to a feature extraction portion (and/or additional portions) of a next network in the pipeline until a final, trained network is provided that can improve analysis of a relatively small dataset, for example, a dataset comprising a cohort of interest including candidates for, or participants in, a clinical trial of a new treatment.

In a broad sense, embodiments of the present disclosure illustrate a training pipeline comprising successive training of successive deep learning (or other machine learning) networks using successive data sets from less relevant to more relevant datasets with respect to a cohort of interest to improve predictions that assess or are relevant to assessing whether a particular patient is likely to respond to a new treatment. In some embodiments, certain pre-processing techniques are used to improve performance in the context of particular histopathology applications.

In various embodiments, subjects predicted to exhibit altered genotypes can undergo additional molecular testing to confirm the in silico results. In various embodiments, once confirmed through the molecular testing, appropriate medical treatment or medical advice can be provided to subjects based on the altered genotype status. In various embodiments, once confirmed, these subjects can be deemed eligible for enrollment in a clinical trial. In various embodiments, subjects predicted to exhibit wildtype genotypes are likely ineligible for the clinical trial and need not undergo further molecular testing, thereby saving resources (e.g., time and money). In various embodiments, the methods and/or non-transitory computer readable media described herein can operate as a Software as a Medical Device (SaMD) that is provided either on disk or via download or as a web-based software. In such embodiments, the methods described herein can operate independent of a clinical trial setting.

Further aspects of these and other embodiments are described herein.

### BRIEF DESCRIPTION OF THE DRAWINGS

FIG. 1 illustrates a computerized pipeline system for developing a trained computerized deep learning network to make genotype predictions and treatment response predictions using histopathology images corresponding to a cohort of interest including patients in, or candidates for being in, a clinical trial of a new treatment.

FIG. 2 is a block diagram illustrating the architecture of a self-supervised deep learning network of the embodiment of FIG. 1.

FIG. 3 is a block diagram illustrating the architecture of an attention-based learning network of the embodiment of FIG. 1.

FIG. 4 illustrates further details of pre-processing of histopathology image data of one example.

FIG. 5 illustrates further details of the augmentation processing of histopathology image data implemented by the self-supervised learning network illustrated in FIG. 2.

FIG. 6 illustrates further details of a feature extraction network and a projection network of the self-supervised learning network illustrated in FIG. 2.

FIG. 7 illustrates a computer-implemented method for providing final, trained deep learning network to predict tumor genotypes and/or predict responses in members of a cohort of interest to a new treatment using a pipeline system such as the system of FIG. 1.

FIG. 8 shows an example of a computer system 7000, one or more of which may be used to implement one or more of the apparatuses, systems, and methods illustrated herein.

FIG. 9 depicts a system environment overview for determining a genotype prediction for a subject, in accordance with some embodiments.

FIG. 10 depicts a block diagram of the genotype prediction system referenced in FIG. 9.

FIG. 11 depicts an example flow diagram for determining a genotype prediction for a subject, in accordance with some embodiments.

FIG. 12 is an example flow process for determining a genotype prediction for a subject, in accordance with one embodiment.

FIG. 13 illustrates an example computer for use in implementing various embodiments shown herein.

FIG. 14 depicts an example process for generating a patient-level genotype prediction using slide images.

FIG. 15 depicts another example process for generating a patient-level genotype prediction using slide images.

FIG. 16A shows lack of cross-cohort generalizability as TCGA and Tempus samples are distinguished.

FIG. 16B shows the neural network's bias towards pen artifacts that differentiates the TCGA and Trial #1 samples when a pre-processing step is not implemented.

FIG. 16C shows the neural network's improved treatment of TCGA and Trial #1 samples when a pre-processing step is implemented.

FIG. 17A depicts the performance of a model deployed in the example process of FIG. 15 in comparison to a model deployed in the example process of FIG. 14.

FIG. 17B describes performance of a model deployed on TCGA bladder cancer slides according to the example process of FIG. 15.

FIG. 18 depicts another example process for generating a patient-level genotype prediction using slide images.

FIG. 19 depicts the performances of a model deployed in the example process of FIG. 18 in comparison to a model deployed in the example process of FIG. 14.

FIG. 20 shows respective heatmaps of the example process described in FIG. 5 and the example process described in FIG. 18, demonstrating that the example process of FIG. 18 is more robust to image artifacts, such as pen marks.

FIG. 21 depicts another example process for generating a patient-level genotype prediction using slide images.

FIG. 22 depicts an example pipeline workflow including several quality control steps to improve the percentage of high quality images that are analyzed by the neural network.

While the embodiments are described with reference to the above drawings, the drawings are intended to be illustrative, and other embodiments are consistent with the spirit, and within the scope, of the disclosure.

### DETAILED DESCRIPTION

The various embodiments now will be described more fully hereinafter with reference to the accompanying drawings, which form a part hereof, and which show, by way of illustration, specific examples of practicing the embodiments. This specification may, however, be embodied in many different forms and should not be construed as limited to the embodiments set forth herein; rather, these embodiments are provided so that this specification will be thorough and complete, and will fully convey the scope of the disclosure to those skilled in the art. Among other things, this specification may be embodied as methods or devices. Accordingly, any of the various embodiments herein may take the form of an entirely hardware embodiment, an entirely software embodiment or an embodiment combining software and hardware aspects. The following specification is, therefore, not to be taken in a limiting sense.

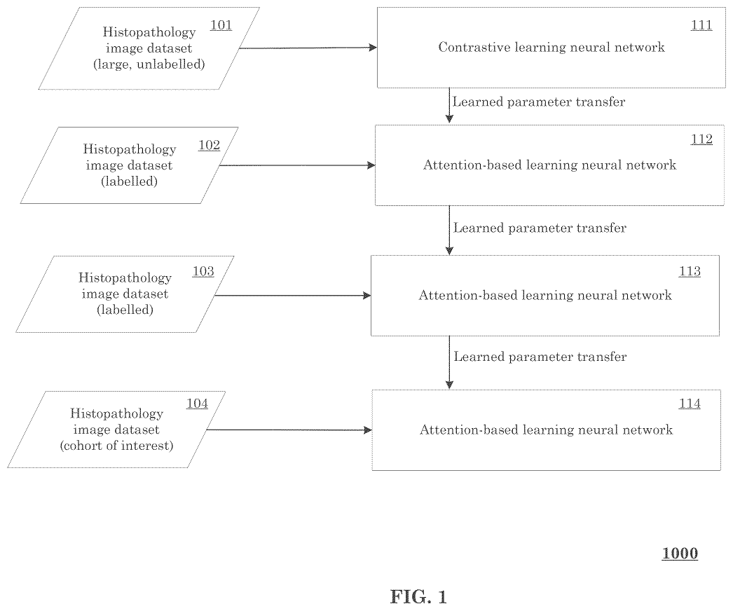

FIG. 1 illustrates a computerized pipeline system 1000 for developing a trained computerized deep learning network (or other computerized machine learning model) to make cancer genotype predictions, treatment response predictions, and/or other predictions using histopathology images corresponding to a cohort of interest, especially, for example, a cohort of interest including patients in, or candidates for being in, a clinical trial of a new treatment (or of a new combination of treatments).

FIG. 1 is illustrated and described using the example of a series of neural networks 111, 112, 113, and 114. However, the underlying principles are applicable to other deep learning or machine learning computer systems, whether or not those systems include any neural network processing layers. A “neural network” in this example, simply means a computerized system that implements machine learning processing including one or more processing layers or elements known as neural network layer or elements. In various examples, this might include feed forward neural network layers (also known as fully-connected layers), convolutional neural network layers (which may or may not include residual connections), recurrent neural networks, or other types of neural network layers or elements. Such computerized machine learning systems are referred to herein as “neural networks” if the system includes any processing layer or element known to be a type of neural network processing layer or element. Even if such a system has many additional components or layers other than the neural network elements or layers, it will be referred to herein as a “neural network.” Again, however, those skilled in the art will understand that certain inventive principles of the illustrated embodiment are applicable to other types of machine learning systems embodying the disclosure, even if those systems do not include any neural network components.

Specifically, pipeline system 1000 includes a series of respective neural networks (which could also be referred to as neural network modules, deep learning modules, and/or machine learning modules) 111, 112, 113, and 114. Respective histopathology image datasets 101, 102, 103, and 104 are used, in succession, to respectively train neural networks 111, 112, 113, and 114. In this example, the pipeline shows four neural networks in the pipeline system. However, in alternative implementations, more or fewer neural networks (or other types of machine learning modules), and more or fewer datasets could be used.

Pipeline system 1000 is tailored for use in situations where the histopathology image dataset of highest relevance, 104, is necessarily relatively small as it relates to a cohort of interest typically including participants (or candidates for participation) in a clinical trial for a new therapeutic treatment for a particular disease, for example, administering a new drug, a new combination of drugs, and/or a using new protocol for treating the relevant disease. For example, a small dataset such as dataset 104 might include images from only 100-200 patients. In many examples, it might even correspond to histopathology images from 100 patients or less than 100 patients (e.g., 40-50 patients, 51-60 patients, 61-70, 71-80, 81-90, 91-100 patients).

The challenge posed by such small histopathology image datasets is that it is generally too difficult or not possible to effectively train a deep learning network from scratch to make sufficiently accurate predictions using such a small amount of training data. However, it is possible to use such a small dataset to fine-tune/train a deep learning network that has a feature extraction portion that has been pre-trained (or has weights that have been transferred from an appropriately pre-trained network) on other datasets, preferably, a series of datasets starting with large, less relevant datasets and continuing with increasingly relevant (and typically smaller) datasets.

In the illustrated example, dataset 101 is a large, publicly available dataset. For many diseases, such as cancer, there are large publicly available datasets (e.g., The Cancer Genome Atlas-“TCGA”-data) that might correspond to histopathology images from more than 20,000 or even more than 30,000 individuals. Although such datasets will typically identify the type of cancer, they will not necessarily also identify other specific information that might be needed for using fully supervised learning for predicting responses to various treatments. Thus, such datasets might be “unlabeled” in the sense that specific known information regarding treatment results or even identifying specific tumor genotypes within a specific type of cancer (e.g., lung), might not be available.

Although a dataset 101 might be labeled for some purposes, in the specific example described herein, it is not labeled for the specific purpose of training a treatment response classifier (or if it does have treatment response information, the corresponding treatment and/or cancer information is not relevant enough to the cohort of interest to use the labels for useful training purposes).

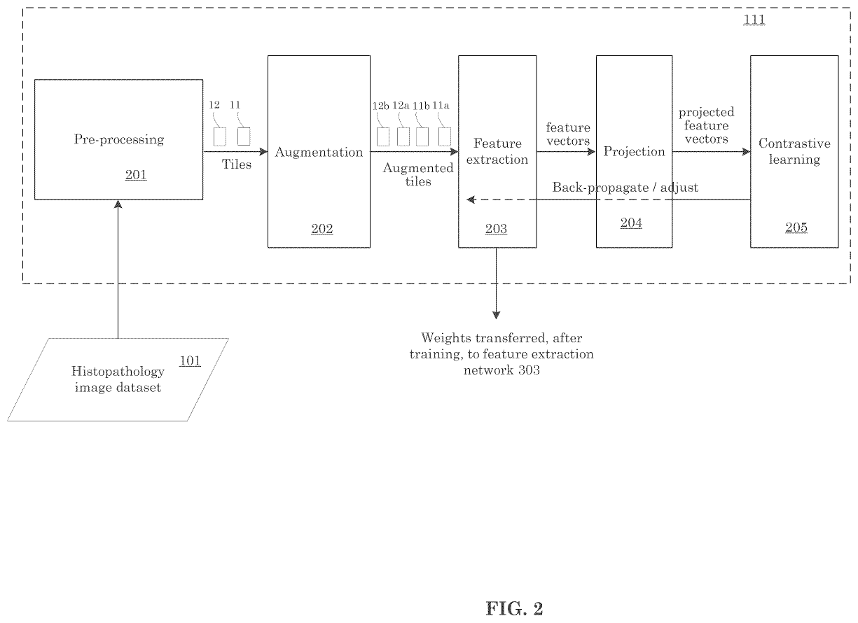

However, even if dataset 101 is not labeled (or otherwise does not have associated information that is sufficiently relevant enough for fully supervised learning), it can be effectively used for self-supervised learning to, for example, pre-train a feature extractor to extract relevant features from histopathology image (or other medical image) in the context of cancer or another other disease. Therefore, in pipeline system 1000, first deep learning network 111 performs self-supervised learning (which does not require the use of labeled training data) to begin training a feature extraction portion of deep learning network 111 using dataset 101. As further described in the context of FIG. 2 below, in this example, deep learning network 111 is configured to implement contrastive learning. However, in other implementations consistent with the principles of this disclosure, a first deep learning network in the pipeline might be configured to implement other types of self-supervised learning.

At least a portion of the learned parameters (sometimes referred to as weights, kernel values, filter values, or other names) that result from conducting self-supervised learning using contrastive learning neural network 111 and histopathology image dataset 101 are transferred to second network 112. In this example, network 112 is an attention-based multi-instance deep learning network.

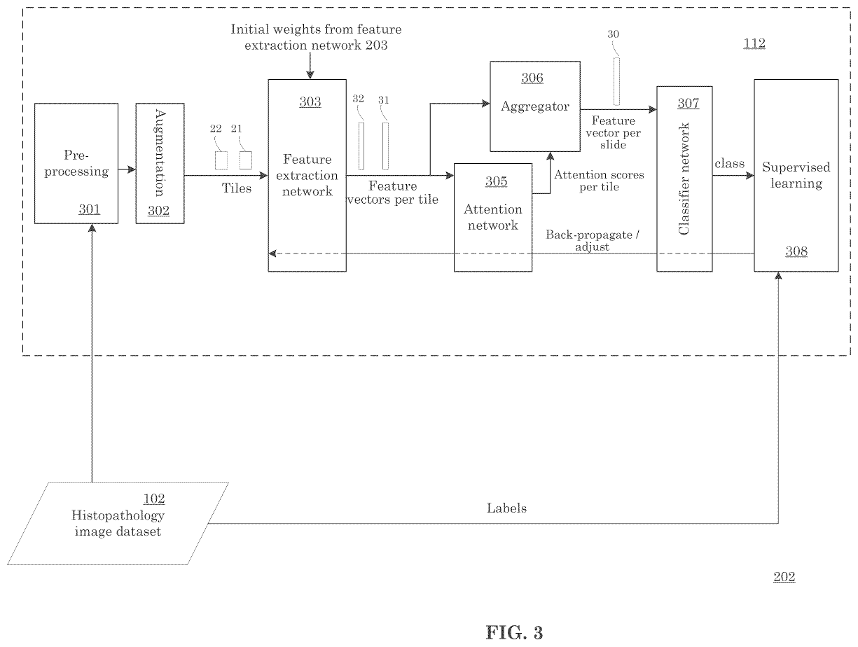

In pipeline system 1000, second network 112 is trained by histopathology image dataset 102. In this example, histopathology image dataset 102 is a labeled dataset and network 112 carries out attention-based supervised learning using histopathology image dataset 102 as described further in the context of FIG. 3.

In the illustrated example, dataset 102 is a smaller and more relevant dataset than dataset 101 with more corresponding relevant information. For example, it might be a commercial dataset (i.e., private) that includes treatment outcomes and/or molecular testing results identifying genotypes for specific mutations relevant to a particular cancers corresponding to tissues in the histopathology images. It might be in the context of the same disease or a similar disease as the disease corresponding to the cohort of interest, but the treatment and other aspects surrounding the data are different. After second network 112 is trained using histopathology image dataset 102, at least some of the resulting learned parameters are transferred to third deep learning network 113, which, in this example, is also an attention-based multi-instance deep learning network.

In pipeline system 1000, third network 113 is trained by histopathology image dataset 103. In this example, histopathology image dataset 103 is a labeled dataset corresponding to a clinical cohort that is not the cohort of interest, but that is more relevant to the cohort of interest than is dataset 102. In one example, histopathology image dataset 103 corresponds to a clinical trial cohort that has received a treatment that has some relevance to the treatment to be administered to the cohort of interest, but is not the same treatment. In this example, the clinical trial corresponding to dataset 103 has similar inclusion/exclusion criteria as the clinical trial for the new treatment to be administered to the cohort of interest.

After third network 113 is trained using histopathology image dataset 103, at least some of the resulting learned parameters are transferred to a final deep learning network 114, which, in this example, is also an attention-based multi-instance deep learning network.

Final deep learning network 114 is fine-tuned (further trained) using histopathology image dataset 104 which corresponds to at least a portion of the cohort of interest whose members are in, or are candidates for being in, a clinical trial of the new treatment. In one example, histopathology image dataset 104 corresponds to a portion of the cohort of interest that has already received the new treatment and some data regarding response to that treatment is available.

After fine-tuning (i.e., training) of final network 114 using histopathology image dataset 104, network 114 can then be used to assist in predicting treatment responses for other members of the cohort of interest using histopathology images that correspond to those other members of the cohort of interest. In this manner, a final network such as network 114 that has been developed using pipeline system 1000 can allow for improved selection of participants in the relevant ongoing clinical trial or in future clinical trials involving the same or similar treatment.

The illustrated example of a pipeline system is described herein in terms of relevant learned weights being transferred from one deep learning network to a next deep learning network in the pipeline. However, one skilled in the art will understand that, in some instances (for example, when successive networks in the pipeline have the same architecture), this can be considered equivalent to successively training the same network first with one dataset and then with another dataset or, as another example, successively training the same network first on one data set and then re-initializing weights of some parts of the network (e.g., attention and classification layers, in which weights from training on a prior dataset are not retained when starting training on a the next data set in the pipeline) but not others (e.g., feature extraction layers, in which weights from training on a prior dataset are retained as starting values for training on the next dataset) when beginning training with next dataset. Such examples are considered consistent with the spirit and scope of the present disclosure.

FIG. 2 is a block diagram illustrating the architecture of self-supervised learning neural network 111 (one example of a deep learning network consistent with a first deep learning network in a pipeline system embodying the underlying principles of the present disclosure) of the pipeline system 1000. Specifically, network 111 comprises pre-processing block 201, augmentation block 202, feature extraction network 203, projection network 204, and contrastive learning module 205.

Contrastive learning network 111 illustrated in FIG. 2 is similar to the contrastive learning network disclosed in applicant's co-pending U.S. Provisional Application No. 63/301,023 filed on Jan. 19, 2022 and hereby incorporated by reference in its entirety. Contrastive learning network 111 of the present disclosure is adapted to process data from histopathology images. In one embodiment, the system operates to train feature extraction neural network 203 and projection neural network 204 (network 203 and network 204 can also be thought of as simply different sets of layers of a single neural network) to extract features from histopathology images that will enable effective classification of (or other supervised learning tasks regarding) tissue samples captured in histopathology images.

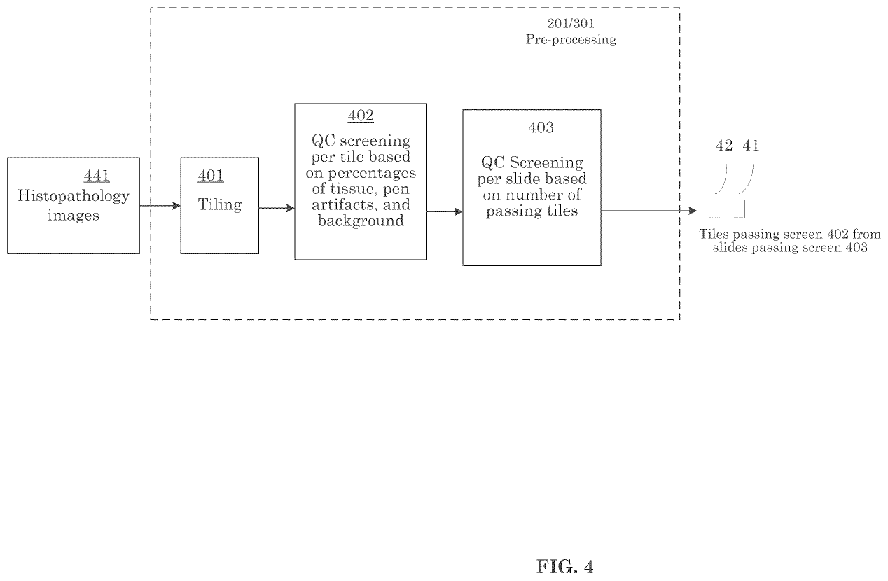

Pre-processing module 201 receives data from histopathology image dataset 101 and preprocesses it to provide tiles such as tile 21 and tile 22, to augmentation module 202. Additional details of pre-processing module 201 will be discussed in the context of FIG. 4. Tiles 22 and 21 are pre-processed pixel data corresponding to a portion of a histopathology image from dataset 101.

Pre-processing module 201 receives histopathology image data from histopathology image dataset 101. Histopathology images from dataset 101 are typically digital images of stained (dyed) tissue samples which may be obtained, for example, from biopsies of patients suspected of having a particular disease. Pre-processing module 201 divides each histopathology image into a plurality of tiles, also referred to herein as patches.

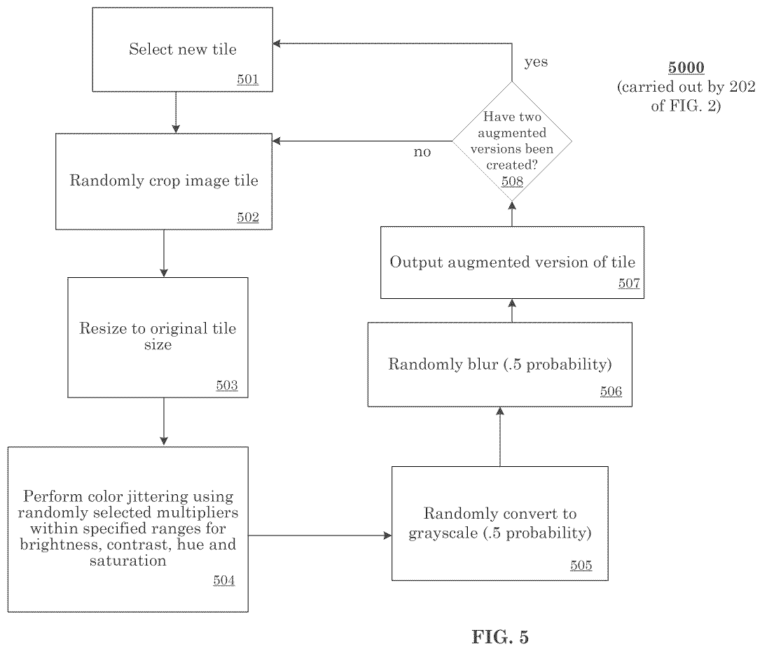

Augmentation module 202 receives the pre-processed tiles and may perform two different executions of an augmentation process on each tile to generate two different augmented versions of each tile. For example, in the illustrated example, augmentation module 202 receives two different tiles 21 and 22 and, from tile 21, generates augmented tile 21a and augmented tile 21b, corresponding to two different iterations of an augmentation process, and, in similar fashion, from tile 22, generates augmented tile 22a and augmented tile 12b, also corresponding to two different iterations of the augmentation process. In one example, an augmentation process might include a series of steps performed on a tile such as: random cropping followed by resizing to the original size; random horizontal and/or vertical flipping; color jittering using randomly selected multipliers within a specified range for brightness, contrast, hue, and saturation; and/or randomly converting the image tile to grayscale (e.g., with a 0.5 probability). Such a process could be performed twice on the same tile (e.g., tile 21) to generate two different augmented versions of the tile (e.g., 21a and 21b). One example of a particular augmentation process is shown in detail in FIG. 5.

Returning to the description of FIG. 2, in the illustrated example, two augmented tile pairs are generated from each tile fed into augmentation module 202. However, additional augmented tile versions can be generated from each tile.

Augmented tiles are processed by feature extraction network 203. Feature extraction network 203 may be based on the convolutional layers and the average pooling layer of ResNet 34, a 34-layer residual network as described in He et al., Deep Residual Learning for Image Recognition, available at arXiv:1512.03385v1, 10 Dec. 2015, incorporated herein by reference (“ResNet Paper”). The classification layers (fully connected layers and softmax layer) of ResNet 34 are not utilized, but are replaced by projection network 204. However, this is just an example. Different size and/or types of feature extraction networks can be used consistent with the principles of the present disclosure.

Feature extraction network 203 provides feature vectors (one per augmented tile) to projection network 204. Projection network 204 (further described below in the context of FIG. 4) processes the feature vectors and provides projected feature vectors to contrastive learning module 205.

Contrastive learning module 205 applies a loss function to compute a loss measure for features vectors corresponding to processed tiles in a same batch. The loss measure can be related differences between feature vectors derived from different augmented versions of the same tile in the batch and is also related to differences between feature vectors corresponding to augmented versions of different tile in the batch. The loss is then back propagated through projection network 204 and feature extraction network 203 and used to adjust learnable parameters (weights) of those networks. To put it simply, as the system learns to produce better feature vectors, it decreases the difference between feature vectors from different augmented versions of the same tile while increasing the difference between feature vectors from augmented versions of different tiles.

Contrastive learning module 205 may implement the “SimCLR” contrastive learning processing set forth in Chen et al., A Simple Framework for Contrastive Learning of Visual Representation, Proceedings of the 37^{th} International Conference on Machine Learning, Vienna, PMLR 119, 2020, incorporated herein by reference (“SimCLR Paper”). Specifically, the NT-Xent loss referenced therein may be used as the loss function of contrastive learning module 205. In a particular example, a loss temperature of 0.1 is used. In alternative examples, other loss temperatures can be used. Also, in alternative examples, other loss functions can be used.

In a particular example, a batch size of 768 is used. In alternative examples, smaller or larger batch sizes are used. In other examples, batch size is in the range of about 250-4,000. Also, in one example, an Adam optimizer is used and an unscheduled learning rate of about 5×10⁻⁴ is used.

FIG. 3 is a block diagram illustrating the architecture of attention-based learning network 112 (one example of a deep learning network consistent with a second deep learning network in a pipeline system embodying the underlying principles of the present disclosure) of the pipeline system 1000. Specifically, network 112 comprises pre-processing block 301, augmentation module 302, feature extraction network 303, attention network 305, aggregator 306, classifier network 307 and supervised learning module 308.

In the illustrated example, pre-processing block 301 is substantially similar to pre-processing block 301 of FIG. 2 and is further discussed below in the context of FIG. 4.

In one example, augmentation module 302 implements different and/or fewer augmentation steps than implemented by augmentation module 202 in FIG. 2. For example, augmentation module 302 might implement only color jittering (also known as stain based augmentation), but not implement other augmentation steps. In other examples, the same augmentations might be used. In other examples, some augmentation steps implemented by augmentation module 302 are different than any of the steps of augmentation module 202. Those skilled in the art will recognized that, in the context of attention-based learning networks 112-114 (or other deep learning networks), augmentation is potentially useful for enhancing training robustness. However, the same augmentation steps used in the context of self-supervised learning are not necessarily needed or optimal for attention-based supervised learning. And in some examples, augmentation may or may not be used during supervised learning without necessarily departing from the spirit and scope of the present disclosure. In the illustrated embodiment, although augmentation is applied to training data used during training attention-based networks 112-114, when a final, fully trained network is applied to non-training histopathology image data for making predictions to assist decision-making, augmentation processing is not applied to the analyzed histopathology image data.

In the illustrated example, the details of feature extraction network 303 are substantially the same as those of feature extraction network 203 in FIG. 2 as described above. However, the architecture of those blocks could be different for different steps in a deep learning pipeline consistent with the present disclosure. For example, if a first data set used to train a first network in a pipeline is different (e.g., different type of images, different data dimensions, etc.) than a second data set used to train a next network in the pipeline, then the pre-processing and/or feature extraction portions of each network might be different.

In the example illustrated above, weights from feature extraction network 203 learned in training deep learning network 111 are transferred to feature extraction network 303 prior to training deep learning network 112 using data set 102. In the current example, feature extraction networks in each deep learning network 111, 112, 113, and 114 have the same architecture and dimensions. So weights learned in training each network in the pipeline can be transferred to provide all of the initial weights the feature extraction portion of the next deep learning network in the pipeline. However, even if feature extraction networks are different from one pipeline network to another, weights from a prior network in the pipeline can be transferred to comparable positions in a next network in the pipeline and, for example, any other positions in the next network can be initialized at zero or at some other value prior to training the next network. If needed, weights can be further processed during transfer via other techniques such as averaging, interpolation, or other methods so that the benefit of training feature extraction portions of a prior network can benefit training of the next network in the pipeline.

Continuing with the description of FIG. 3, feature extraction network 303 receives pre-processed tiles such as tiles 21 and 22 and processes them to extract feature vectors for each tiles such as feature vectors 31 and 32. Feature vectors for each tile are fed to attention network 305 and to aggregation function 306. In this example, attention network 305 is simply a feed forward neural network comprising one or more fully connected layers with corresponding weights. The input layer size corresponds to the feature vector size and the output layer provides a single attention value for each tile. The output score (attention value) is normalized to be no less than 0 and no greater than 1.

Aggregator 306 multiplies each tile's feature vector by its corresponding attention score and then averages all feature vectors for a given histopathology image of a patient to produce a summarized feature vector 30 such that one summarized feature vector is produced for each patient's histopathology image. In the current example, summarized feature vector 30 will have the same dimensions as a feature vector for an individual tile. E.g., summarized feature vector 30 will have the same dimensions as single tile's feature vector such as feature vector 31. But in alternative examples, the sizes of summarized feature vectors do not necessarily exactly match the size of feature vectors for individual tiles.

Summarized feature vectors (one for each patient's histopathology image) are then provided to classifier network 307 (which, in this example, includes a typical feed forward network of one or more full connected layers) which produces a prediction value (or class) for each summarized feature vector. Supervised learning module 308 uses a loss function to compute a loss value based on a label corresponding to the relevant histopathology image in dataset 102 and based on the class value provided by classifier network 307. Depending on the prediction type, different loss functions can be used. For example, if the prediction is in the form of survival time, or time to recurrence (which can be one of many values, e.g., a number of weeks), a cross-entropy loss function can be used. However, if the prediction is binary (e.g., response/no response to treatment; or presence/non-presence of a particular mutation), then a binary cross-entropy loss function can be used. These are just examples. Various loss functions for supervised learning are within the capability of one skilled in the art and a particular loss function used is not intended to limit the broader aspects of the present disclosure.

Supervised learning module 308 then back propagates the loss value (sometimes referred to as error value) through classifier network 307, attention network 305, and feature extraction network 303 and uses it to adjust weights (learnable parameters) in those networks. Various known techniques for backpropagation and weight adjustment can be used and learning rates, and other learning parameters can be selected and modified to enhance performance for a particular application.

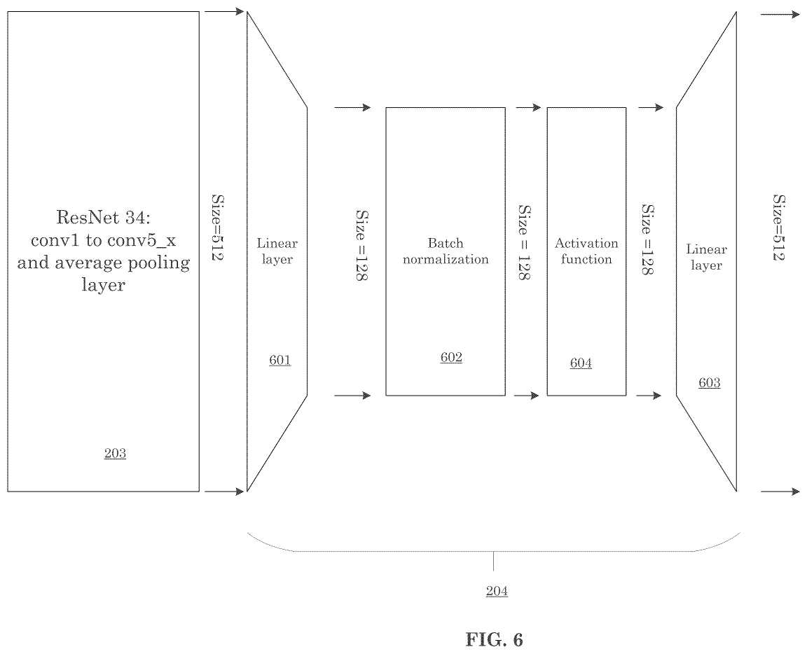

In one example, the overall architecture of deep learning network 112, after pre-processing block 301, is based on aspects of the attention-based multi-instance deep learning architecture shown in Isle et al., Attention-based Deep Multiple Instance Learning, published at arXiv:1802.04712v4 [cs.LG] 28 Jun. 2018 (referenced herein as “the attention-based MI learning paper”) and incorporated herein by reference in its entirety (see, e.g., FIG. 6c of that paper's appendix). However, this is just one example. In other examples, different network architectures might be used for different networks in a pipeline system consistent with the present disclosure (e.g., other attention-based architectures or types of deep learning networks other than attention-based networks).

In the specific example of pipeline system 1000 of FIG. 1, the architecture of third network 113 and final network 114 are the same as that shown for second network 112 illustrated in FIG. 3 and will not be separately discussed herein. However, note that the architecture of pipeline networks from a second network to a last network need not be identical.

Furthermore, in the specific example of pipeline system 1000 of FIG. 1, only the weights from feature extraction networks such as feature extraction network 303 are transferred from one network in the pipeline to another. For example, as those skilled in the art will appreciate, during training of network 112 shown in FIG. 3, weights will be learned in attention network 305 and classifier network 307 as well as in feature extraction network 303. However, in the present example, only the weights in feature extraction network 303 will be transferred to a feature extraction network of the next network in the pipeline (e.g., network 113 of FIG. 1). Weights for attention and classifier networks in subsequent deep learning networks in the pipeline will be re-initialized and retrained from scratch. However, weights from the feature extraction network will be transferred. Nevertheless, in alternative examples, feature extraction network weights and other weights (such as, for example, weights from classification network such as classifier 307) could be transferred to a next network in the pipeline prior to training the next network without necessarily departing from the principles of the present disclosure.

FIG. 4 illustrates further details of pre-processing block 201 of FIG. 2 (which are the same as details of pre-processing block 301 of FIG. 3). Specifically, pre-processing block 201 comprising tiling module 404, quality control (QC) screening per tile module 402, and QC screening per slide module 403 (“slide” is used interchangeably herein with “histopathology image” corresponding to a particular tissue sample).

Tiling module 401 receives histopathology images 441 (from a histopathology data set such as dataset 101) and divides the image into a plurality of tiles (also sometimes referred to as “patches” herein). In one particular embodiment, an image is divided into non-overlapping tiles, each tile being 224 pixels by 224 pixels in size. However, other sizes can be used in alternative examples.

QC screening per tile module 402 processes each tile to identify image quality. In one example, image quality for a tile is determined by determining the percentage of tissue in the tile relative to background and relative to artifacts such as pen marks. One example of processing to do this is provided at: https://github.com/CODAIT/deep-histopath/tree/master/deephistopath/wsi. In one example, the following processing steps are performed by module 402 for each tile: 1. obtain a low-resolution thumbnail; 2. compute a pen mask (e.g., based on identifying RGB pen colors) and a background mask (e.g., based on identifying “off-colors” such as greys and greens that are not H&E stain colors); 3. remove computed mask from slide; and 4. at high resolution, for each tile location a) obtain pen mask and background mask and apply to low resolution version of tile, b) compute percentages of tissue/pen/background in tile, c) estimate color measurements, d) use tissue percentage and color measurements to compute a QC score. In one example, a QC score is given by the following formula:

$$QC score = tissue {percentage}^{2} \times \frac{ln ( 1 + c olorfactor + sautrationvalue factor + quantityfactor )}{1 , 000}$$

In the above formula, the “colorfactor” is based on H&E stain colors and weights hematoxylin higher than eosin; “saturationfactor” is based on the fact that real tissue has broad HSV saturation, and “quantityfactor” corresponds to the amount of tissue in the tile image. In one example, a QC score using the above formula is scaled to be between 0 and 1 and a tiles below a threshold score are rejected. In one example, the threshold is 0.75. In other examples, it is higher or lower than 0.75 (e.g., a number between 0.6-0.75; a number between 0.75-0.95; etc.).

QC screening per slide module 403 determines whether a sufficient number of tiles corresponding to a particular slide (i.e., a particular tissue image) pass the QC screening 402. In one example, if an insufficient number of tiles pass screen 402, then module 403 discards all tiles from that slide. Pre-processing 201/301 output tiles such as 42 and 41 that have passed screen 402 and are from images that have not be rejected by screening 403. The illustrated processing is just one example of a QC screening process. Other QC screening processes can be employed to determine tiles and slides with sufficient tissue imaging to be usefully employed for further analysis without necessarily departing from the present disclosure.

FIG. 5 show a particular example of augmentation processing 5000 that may be carried out by augmentation module 202 of FIG. 2 in the context of self-supervised learning. The inventors have found that the parameters of the illustrated embodiment work particularly well for implementing contrastive learning with histopathology slides. However, other processing and parameters can be used without necessarily departing from the present disclosure.

Step 501 selects a new tile for augmentation. Step 502 randomly crops the image tile. Step 503 resizes it to its original size, in this example, 224×224 pixels. Step 504 randomly modifies the color parameters of the tile (this action is referred to as “color jitter” in the context of machine vision). In one example, randomly selected values in pre-determined ranges are applied to the image for brightness, contrast, saturation, and hue. In one example, the pre-determined ranges from which values for brightness, contrast, and hue are randomly selected are each about 0.2 to 1.8 and the pre-determined range from which a value for hue is randomly selected is about −0.2 to 0.2.

Step 505 then randomly converts the tile output by step 504 to grayscale. In this example, the random function uses a 0.5 probability of conversion. I.e., the tile has about a 50% chance of being converted to grayscale at this step. In other examples, other probabilities are used.

At step 506, the output of step 505 is randomly blurred. In a particular embodiment, a 0.5 probability is used to determine whether the tile is randomly blurred during augmentation.

Step 507 outputs an augmented version of the current tile. At step 508, if two augmented versions of the same tile have not yet been created, the method proceeds to step 502 to perform another iteration of augmentation steps 502-506 on the same tile. However, if two augmented versions of the same tile have already been output, then the method returns to step 501 and selects a new tile for augmentation.

FIG. 6 illustrates further details of feature extraction network 203 and projection network 204 of FIG. 2. Specifically, in the illustrated example, feature extraction network 203 uses the convolutional layers associated with conv1, conv2_x, conv3_x, conv4_x, conv5_x, and the average pooling layer, but NOT the fully connected layers of ResNet 34 in the ResNet Paper. It also uses all skip connections associated with those layers in the ResNet Paper. In this example, feature extraction network 203 outputs feature vectors having 512 values to projection network 204.

Projection network 204 may comprise linear layer 601, batch normalization layer 602, activation layer 604, and linear layer 603. Linear layer 601 comprises an input layer and a fully connected hidden layer of 128 neurons (without activation functions). Thus linear layer 601 outputs a feature vector of size 128 to batch normalization layer 602. Batch normalization layer 602 performs standard batch normalization processing. After passing through batch normalization layer 602, the feature vector passes through activation function layer 604 implementing a non-linear activation function such as ReLu and then to linear layer 603 which comprises an input layer of size 128 and a fully connected hidden layer of 512 neurons (without activation functions), and which therefore projects the feature vector back up to 512 in size.

It will be understood that, in this context, “projection network” simply refers to the fact that the feature representations undergo changes in the number of dimensions used for representation as they are passed through the network. In this example, the projected features have the same dimensions as the input features but have been obtained through a process in which the feature representations were first projected into a lower dimensional representation and then, after batch normalization, projected back up into a representation having the same number of dimensions as the input representations. Alternatively, use of a projection network may be omitted such as that illustrated and described herein, better results are obtained by using a projection network and batch normalization before passing resulting feature vectors to a contrastive learning module.

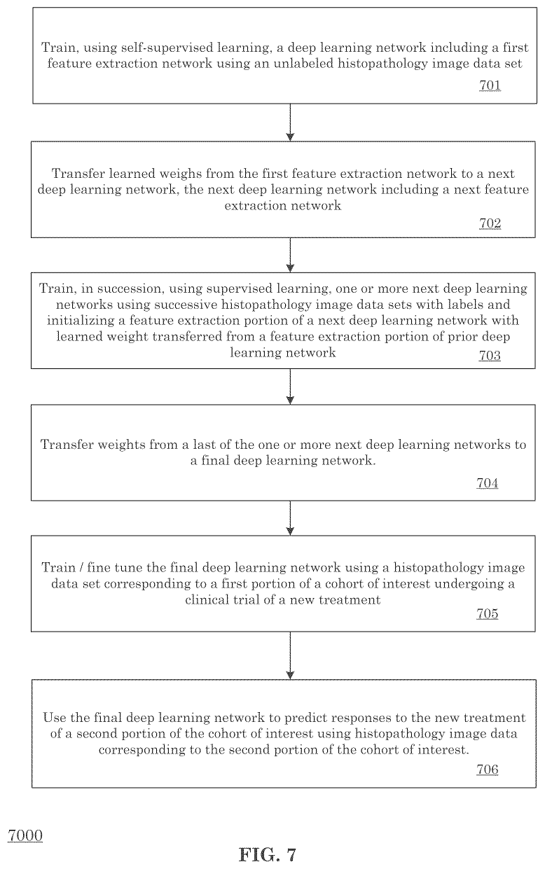

FIG. 7 illustrates a computer-implemented method 7000 for providing final, trained deep learning network to predict responses in members of a cohort of interest to a new treatment using a pipeline system such as system 1000 of FIG. 1.

Step 701 includes training a first deep learning network using a first histopathology image dataset. In this particular example, contrastive self-supervised learning is used to train a first feature extractor without requiring use of labels from the training dataset (the first medical image dataset).

Step 702 includes transferring weights of the first feature extractor that are learned from self-supervised training of the first deep learning network to a feature extractor of a next deep learning network in a pipeline training system.

Step 703 includes training, in succession, one or more additional next deep learning networks using successive next medical image data sets and transferring weights from a feature extractor of one network of one network in the pipeline to a feature extractor of a next network in the pipeline. Again, in the illustrated example, only weights of the feature extraction portion of each deep learning network are transferred from one network to the next in the pipeline. However, in alternative examples, weight of other portions of the network (e.g., classification portions) could be transferred as well. As previously discussed, successive data sets in the pipeline are increasingly relevant to the cohort of interest for which the final network in the pipeline is to be trained.

Step 704 includes transferring weights to a final network in the pipeline. Step 705 includes fine tuning the final network using a data from a first portion of the cohort of interest in a clinical trial for the relevant new treatment.

Step 706 includes using the final trained deep learning network to make genotype predictions and/or treatment response predictions based on histopathology images corresponding to members of a second portion of the cohort of interest.

### Results and Implications

In some examples, a prediction made for each histopathology image (at the slide-level) is a presence or absence of a genotype alteration. In various examples, the genotype alterations comprise an alteration of gene corresponding to a fibroblast growth factor receptor (FGFR) gene (“FGFR alteration”). In various examples, the FGFR alteration comprises any of FGFR3 mutation, FGFR3 fusion, or a combination of a FGFR3 mutation and FGFR3 fusion. In various embodiments, the FGFR alteration is any of a p.R248C mutation, a p.G370C mutation, a p.S249C mutation, or a p.Y373C mutation. In various embodiments, the FGFR alteration is any of a FGFR3:TACC3V1 fusion, a FGFR3:TACC3V3 fusion, a FGFR3:BAIAP2L1 fusion, a FGFR2:BICC1 fusion, or a FGFR2:CASP7 fusion. In some examples, an FGFR alteration is in the context of a patient with bladder cancer.

In some examples, the alteration comprises an alteration (for example, a DNA-repair deficiency (DRD)), in one or more of the following genes: BRCA1, BRCA2, BRIP1, CDK12, CHEK2, FANCA, PALB2, RAD51B, RAD54L, RAD21, or SPOP. In some examples, alterations in one or more of these genes is in the context of a patient with prostate cancer. In some examples, the alteration comprises an alteration of a MET gene (which codes for a c-MET protein) (“MET alteration”). In some examples, the MET alteration is in the context of a patient with lung cancer.

Alterations might include one or more of various types of alterations including single nucleotide polymorphisms (SNPs), copy number variations (CNVs), or gene fusions. In various embodiments, the image is a hematoxylin and eosin (H&E) stained histopathology image. In various embodiments, the subject has cancer or is suspected of having cancer.

In some applications, methods include (and/or computer program products include instructions for) determining whether to administer a therapeutic according to at least the determined genotype. In various embodiments, the therapeutic is a FGFR kinase inhibitor. In various embodiments, the FGFR kinase inhibitor is erdafitinib (BALVERSA™). In various embodiments, the therapeutic is a PARP inhibitor. In various embodiments, the PARP inhibitor is niraparib (ZEJULA™). In various embodiments, a final trained neural network model exhibits an area under the receiver operating curve (auROC) performance metric of at least 0.82 on bladder cancer images. In various embodiments, a final trained neural network model exhibits a positive predictive value (PPV) performance metric of at least 0.22 at 100% recall on bladder cancer models in a test dataset with a 14% baseline prevalence for FGFR. In various examples, a final, trained neural network model exhibits an auROC performance metric of at least 0.71 on prostate cancer images. In various embodiments, the neural network model exhibits a positive predictive value (PPV) performance metric of at least 0.14 at 100% recall on prostate cancer images in a test dataset with a 11% baseline prevalence for PARP.

In some applications, the tumor tissue slide from the patient subject comprises lung cancer. In some applications, the genotype relates to an alteration of a mesenchymal epithelial transition factor (MET) gene (a MET gene produces a c-MET protein). In some examples, a final, trained neural network model consistent with the present disclosure exhibits auROC performance metric of at least 0.78+/−0.03 for predicting MET genotypes in the context of non-small-cell lung cancer tissue images. In one example, this performance is for predicting whether a patient has five or more copies of the MET gene. In various embodiments, the therapeutic is a bispecific monoclonal antibody targeting epidermal growth factor receptor (EGFR) and MET. In various embodiments, the bispecific monoclonal antibody is amivantamab (Rybrevant™).

In various embodiments, methods disclosed herein further comprise: reporting one or more tiles of the image that are most strongly associated with the genotype of the subject.

Results regarding other specific predictions relevant to efficient clinical trial candidate selection are discuss further below in the context of other figures.

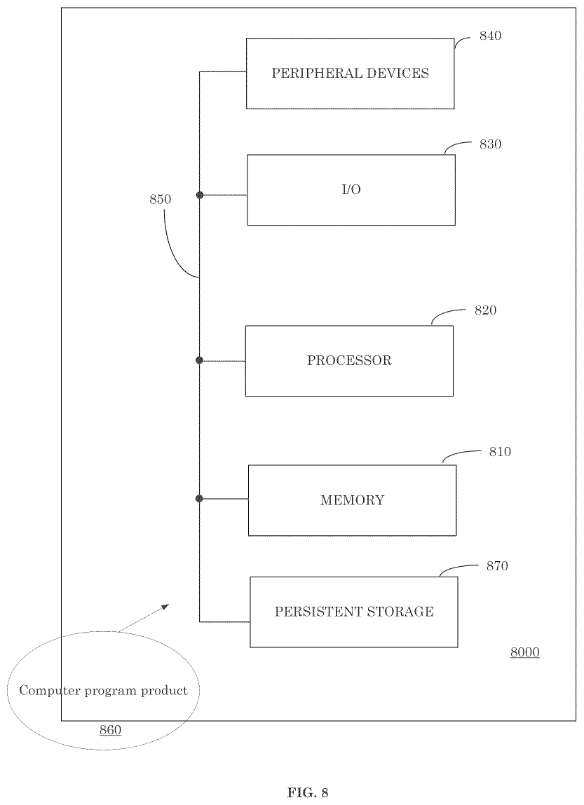

FIG. 8 shows an example of a computer system 8000, one or more of which may be used to implement one or more of the apparatuses, systems, and methods illustrated herein. Computer system 8000 executes instruction code contained in a computer program product 860. Computer program product 860 comprises executable code in an electronically readable medium that may instruct one or more computers such as computer system 8000 to perform processing that accomplishes the exemplary method steps performed.

The electronically readable medium may be any transitory or non-transitory medium that stores information electronically and may be accessed locally or remotely, for example via a network connection. The medium may include a plurality of geographically dispersed media each configured to store different parts of the executable code at different locations and/or at different times. The executable instruction code in an electronically readable medium directs the illustrated computer system 8000 to carry out various exemplary tasks described herein. The executable code for directing the carrying out of tasks described herein would be typically realized in software. However, it will be appreciated by those skilled in the art, that computers or other electronic devices might utilize code realized in hardware to perform many or all the identified tasks. Those skilled in the art will understand that many variations on executable code may be found that implement exemplary methods within the spirit and the scope of the disclosure.

The code or a copy of the code contained in computer program product 860 may reside in one or more storage persistent media (not separately shown) communicatively coupled to system 8000 for loading and storage in persistent storage device 870 and/or memory 810 for execution by processor 820. Computer system 800 also includes I/O subsystem 830 and peripheral devices 840. I/O subsystem 830, peripheral devices 840, processor 820, memory 810, and persistent storage device 870 are coupled via bus 850. Like persistent storage device 870 and any other persistent storage that might contain computer program product 860, memory 810 is a non-transitory media (even if implemented as a typical volatile computer memory device). Moreover, those skilled in the art will appreciate that in addition to storing computer program product 860 for carrying out processing described herein, memory 810 and/or persistent storage device 870 may be configured to store the various data elements referenced and illustrated herein.

Those skilled in the art will appreciate computer system 8000 illustrates just one example of a system in which a computer program product in accordance with the disclosure may be implemented. To cite but one example, execution of instructions contained in a computer program product may be distributed over multiple computers, such as, for example, over the computers of a distributed computing network.

Instructions for implementing an artificial neural network or other deep learning network may reside in computer program product 860. When processor 820 is executing the instructions of computer program product 860, the instructions, or a portion thereof, are typically loaded into working memory 810 from which the instructions are readily accessed by processor 820.

Processor 820 may comprise multiple processors which may comprise respective additional working memories (additional processors and memories not individually illustrated) including one or more graphics processing units (GPUs) comprising at least thousands of arithmetic logic units supporting parallel computations on a large scale. GPUs are often utilized in deep learning applications because they can perform the relevant processing tasks more efficiently than can typical general-purpose processors (CPUs). Processor 820 may additionally or alternatively comprise one or more specialized processing units comprising systolic arrays and/or other hardware arrangements that support efficient parallel processing. Such specialized hardware may work in conjunction with a CPU and/or GPU to carry out the various processing described herein. Such specialized hardware may comprise application specific integrated circuits and the like (which may refer to a portion of an integrated circuit that is application-specific), field programmable gate arrays and the like, or combinations thereof. However, a processor such as processor 820 may be implemented as one or more general purpose processors (preferably having multiple cores) without necessarily departing from the spirit and scope of the present disclosure.

### FURTHER EMBODIMENTS AND SELECTED RESULTS

Terms used in this Further Embodiments and Selected Results section are defined as set forth below unless otherwise specified.

The terms “subject” or “patient” are used interchangeably and encompass a cell, tissue, or organism, human or non-human, male, or female.

The term “mammal” encompasses both humans and non-humans and includes but is not limited to humans, non-human primates, canines, felines, murines, bovines, equines, and porcines.

The term “sample” or “test sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour. In various embodiments, a sample can be a biopsy of a tissue, such as a tumor. In particular embodiments, a sample is a bladder tumor biopsy. In particular embodiments, a sample is a prostate tumor biopsy. In particular embodiments, a sample is a lung tumor biopsy.

The term “obtaining one or more images” encompasses obtaining one or more images captured from a subject or obtaining one or more images captured from a sample obtained from a subject. Obtaining one or more images can encompass performing steps of capturing the one or more images from the subject or from a sample obtained from the subject. The phrase can also encompass receiving one or more images, e.g., from a third party that has performed the steps of capturing the one or more images from the subject or from a sample obtained from the subject. The one or more images can be obtained by one of skill in the art via a variety of known ways including stored on a storage memory.

The phase “subject genotype,” “subject's genotype,” or “subject tumor's genotype” is generally used herein to refer to the genotype of a subject's tumor. In various embodiments, the subject genotype refers to a status of a specific gene, such as a wildtype status or an altered status. For example, the altered status can indicate a presence of a mutation or fusion in the specific gene.

The term “neural network model” refers to a neural network machine learning model. In various embodiments, the neural network model includes multiple submodels (e.g., a first submodel and a second submodel). For example, the neural network model can include a convolutional neural network and an attention network. In other embodiments, the neural network model is composed of only a convolutional neural network.

The terms “treating,” “treatment,” or “therapy” of cancer shall mean slowing, stopping or reversing a cancer's progression by administration of treatment. In some embodiments, treating cancer means reversing the cancer's progression, ideally to the point of eliminating the cancer itself. In various embodiments, “treating,” “treatment,” or “therapy” of lung cancer includes administering a therapeutic agent or pharmaceutical composition to the subject. Additionally, as used herein, “treating,” “treatment,” or “therapy” of cancer further includes administering a therapeutic agent or pharmaceutical composition for prophylactic purposes. Prophylaxis of a cancer refers to the administration of a composition or therapeutic agent to prevent the occurrence, development, onset, progression, or recurrence of cancer or some or all of the symptoms of cancer or to lessen the likelihood of the onset of cancer.

It must be noted that, as used in the specification, the singular forms “a,” “an” and “the” include plural referents unless the context clearly dictates otherwise.

### System Environment Overview

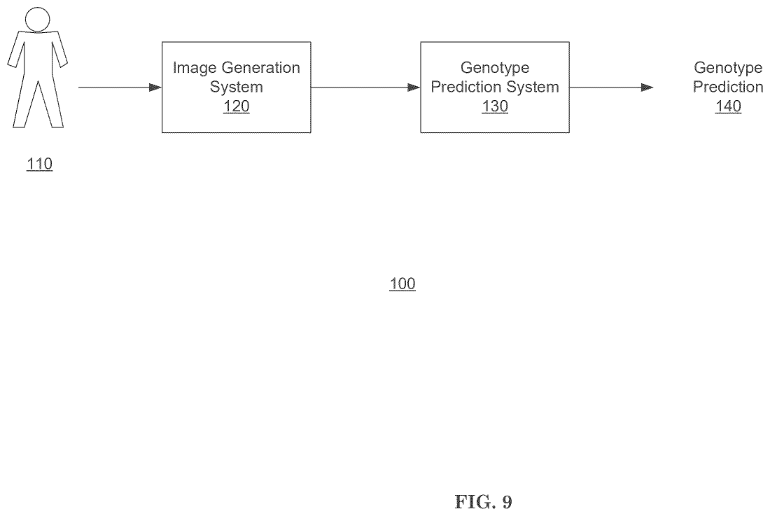

Figure (FIG. 9 depicts a system environment overview for determining a genotype prediction for a subject, in accordance with an embodiment. The system environment 100 provides context in order to introduce a subject 110, an image generation system 120, and a genotype prediction system 130 for determining a genotype prediction 140 for the subject 110. Although FIG. 9 depicts one subject 110 for whom a genotype prediction 140 is generated, in various embodiments, the system environment 100 includes two or more subjects such that that genotype prediction system 130 generates genotype predictions 140 for the two or more subjects (e.g., a genotype prediction for each of the two or more subjects).

In various embodiments, a genotype prediction can be useful for prioritizing subjects for subsequent testing. For example, subjects predicted to exhibit altered genotypes can undergo additional molecular testing to confirm the in silico results. In various embodiments, once confirmed through the molecular testing, appropriate medical treatment or medical advice can be provided to subjects based on the altered genotype status. In contrast, subjects predicting to exhibit wildtype genotypes need not undergo additional molecular testing. Thus, this saves resources as subsets of subjects need not be additionally tested. In various embodiments, a genotype prediction can be useful for determining whether a subject 110 is likely to respond to an intervention. Thus, a subject 110 that is likely to respond to an intervention due to the predicted genotype can be enrolled in a clinical trial in which patients of the clinical trial are to be provided the intervention.

In various embodiments, the subject was previously diagnosed with a cancer, examples of which include bladder cancer or prostate cancer. In various embodiments, the determination of a genotype prediction 140 for the subject can be useful for determining a genotype of interest for the cancer. In various embodiments, the determination of a genotype prediction 140 for the subject can be useful for informing a physician of the likelihood that a patient has a genotype of interest for the cancer. For example, a genotype of interest for bladder cancer is one associated with a gene for fibroblast growth factor receptor (FGFR). As another example, genotypes of interest for prostate cancer include those associated with BRCA1, BRCA2, BRIP1, CDK12, CHEK2, FANCA, PALB2, RAD51B, RAD54L, RAD21, or SPOP. As another example, genotypes of interest for lung cancer include those associated with the MET gene (for c-MET).

In various embodiments, subjects who are predicted to have a particular genotype (e.g., a genotype alteration) can be administered a treatment that slow or prevent the onset, progression, or recurrence of the cancer. In various embodiments, subjects who are predicted to have a particular genotype (e.g., genotype alteration) are selected to be enrolled in a clinical trial. In various embodiments, subjects who are predicted to have a particular genotype (e.g., a genotype alteration) and are subsequently confirmed to have the particular genotype through a molecular test are administered a treatment that slow or prevent the onset, progression, or recurrence of the cancer. In various embodiments, subjects who are predicted to have a particular genotype (e.g., a genotype alteration) and are subsequently confirmed to have the particular genotype through a molecular test are selected to be enrolled in a clinical trial.

Referring to FIG. 9, a test sample is obtained from the subject 110 and the image generation system 120 captures an image from the test sample. In various embodiments, the test sample is a tissue biopsy. For example, the test sample can be a bladder tissue biopsy. For example, the test sample can be a prostate tissue biopsy.

In various embodiments, the test sample is processed to prepare a sample that can be readily imaged by the image generation system 120. In particular embodiments, the test sample is a tissue biopsy that undergoes tissue preparation and a hematoxylin and eosin (H&E) stain such that the image generation system 120 can capture an H&E image of the tissue. For example, a conventional H&E staining process can involve: 1) preserving the tissue biopsy in formalin or paraffin embedding, 2) slicing tissue into thin sections (e.g., 4-5 μm in thickness), 3) removing embedding medium and rehydrating in xylene, ethanol, and deionized water), 4) staining (e.g., antibody staining) for a target, 5) counterstaining using hematoxylin, and 6) mounting of stained tissue slice on a slide for imaging.

The imaging generation system 120 captures an image from the processed sample that is derived from the subject 110. In various embodiments, the image and/or the sample can be obtained by a third party, e.g., a medical professional. Examples of medical professionals include physicians, emergency medical technicians, nurses, first responders, psychologists, phlebotomist, medical physics personnel, nurse practitioners, surgeons, dentists, and any other obvious medical professional as would be known to one skilled in the art. In various embodiments, the image and/or the sample can be obtained in a hospital setting or a medical clinic. In various embodiments, the image and/or the sample can be obtained by a central lab or by a clinical research organization (CRO). In various embodiments, the image and/or the sample can be captured using an imaging device.

In various embodiments, the imaging generation system 120 includes an imaging device, which can be one of a computed tomography (CT) scanner, magnetic resonance imaging (MRI) scanner, positron emission tomography (PET) scanner, x-ray scanner, an ultrasound imaging device, or a light microscope, such as any of a brightfield microscope, darkfield microscope, phase-contrast microscope, differential interference contrast microscope, fluorescence microscope, confocal microscope, or two-photon microscope. In particular embodiments, the imaging device is a light microscope that captures contrast images. In various embodiments, the imaging generation system 120 obtains a contrast image of any one of a bright-field image, phase-contrast image, dark-field image, Reinberg Illumination image, or polarization image.

Generally, the genotype prediction system 130 analyzes one or more images captured from the subject 110 (e.g., images captured by the imaging generation system 120) and generates the genotype prediction 140 for the subject 110. In various embodiments, the genotype prediction 140 determined by the genotype prediction system 130 identifies whether a particular target exhibits a wild type status or an altered status. For example, the genotype prediction 140 can be an indication that a target (e.g., a gene product such as a protein) exhibits a wildtype status. As another example, the genotype prediction 140 can be an indication that a target (e.g., a gene product such as a protein) exhibits an altered status (e.g., the target is altered). In various embodiments, the genotype prediction 140 can be an indication of a specific type of alteration. Examples of a specific alteration can include a presence of a mutation, a presence of a fusion, or a presence of both a mutation and a fusion. As a specific example in the context of bladder cancer, the genotype prediction 140 can be an indication that a subject exhibits a wildtype FGFR status or altered FGFR status. As a specific example in the context of prostate cancer, the genotype prediction 140 can be an indication that a subject exhibits a wildtype BRCA status or altered BRCA status.

In various embodiments, the genotype prediction 140 can be an indication of one of a wildtype status or an altered status, where the altered status is defined by the presence of an alteration in at least one of a plurality of target genes. In various embodiments, the genotype prediction 140 can indicate an altered status based on the presence of an alteration in at least one of at least two genes. In various embodiments, the genotype prediction 140 can indicate an altered status based on the presence of an alteration in at least one of at least three genes, at least four genes, at least five genes, at least six genes, at least seven genes, at least eight genes, at least nine genes, at least ten genes, at least eleven genes, at least twelve genes, at least thirteen genes, at least fourteen genes, or at least fifteen genes. In various embodiments, the genotype prediction 140 can indicate an altered status based on the presence of an alteration in at least one of eleven genes. To provide an example, in the context of prostate cancer, the genotype prediction 140 can be an indication of an altered status based on the presence of an alteration in any one of the following genes: BRCA1; BRCA2; BRIP1; CDK12; CHEK2; FANCA; PALB2; RAD51B; RAD54L; RAD21; SPOP.

In various embodiments, the genotype prediction 140 can include a recommended intervention for the subject 110 based on the predicted genotype status. For example, returning to the context of bladder cancer, if the genotype prediction system 130 determines that the subject 110 exhibits an altered FGFR status, the genotype prediction 140 can include a recommended intervention that is likely to delay the progression, prevent the progression, or reduce the size of bladder cancer exhibiting altered FGFR status.

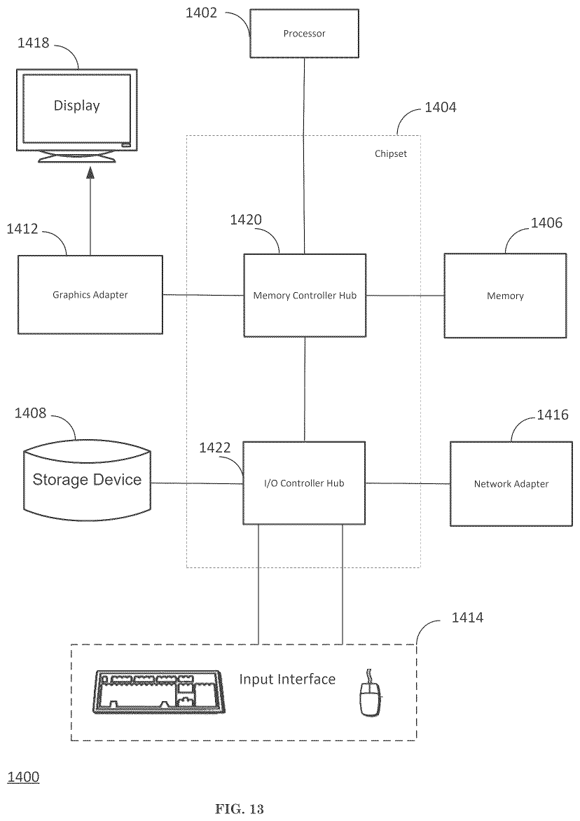

The genotype prediction system 130 can include one or more computers, embodied as a computer system 1400 as discussed below with respect to FIG. 13. Therefore, in various embodiments, the steps described in reference to the genotype prediction system 130 are performed in silico. In various embodiments, the imaging generation system 120 and the genotype prediction system 130 are employed by different parties. For example, a first party operates the imaging generation system 120 to capture one or more images derived from the subject 110 and then provides the captured one or more images to a second party which implements the genotype prediction system 130 to determine a genotype prediction 140. For example, the image generation system 120 can capture the one or more images, stores the one or more images and/or automatically streams the captured one or more images to the genotype prediction system 130 which can automatically analyze the received one or more images to generate genotype predictions 140. In some embodiments, the imaging generation system 120 and the genotype prediction system 130 are employed by the same party.

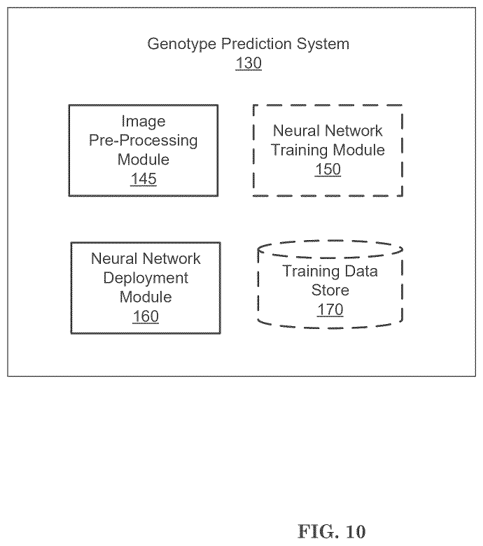

Reference is now made to FIG. 10 which depicts a block diagram of the genotype prediction system, in accordance with an embodiment. Here, the genotype prediction system 130 includes an image pre-processing module 145, a neural network deployment module 160, a neural network training module 150, and a training data store 170. In various embodiments, the genotype prediction system 130 can be configured differently with additional or fewer modules. For example, the genotype prediction system 130 need not include the neural network training module 150 or the training data store 170 (as indicated by their dotted lines in FIG. 10), and instead, the neural network training module 150 and training data store 170 are employed by a different system and/or party.

The components of the genotype prediction system 130 are hereafter described in reference to two phases: 1) a training phase and 2) a deployment phase. More specifically, the training phase refers to the building and training of machine learning models (e.g., neural networks) by the neural network training module 150 based on training data, such as training images captured from training individuals (e.g., individuals whose genotypes are previously known). Therefore, the machine learning models (e.g., neural networks) are trained using the training data such that during the deployment phase, implementation of the machine learning models by the neural network deployment module 160 enables the prediction of genotype for a subject (e.g., subject 110 in FIG. 9).

In some embodiments, the components of the genotype prediction system 130 are applied during one of the training phase and the deployment phase. For example, the neural network training module 150 and training data store 170 are applied during the training phase to train a neural network. Additionally, the neural network deployment module 160 is applied during the deployment phase. In various embodiments, the components of the genotype prediction system 130 can be performed by different parties depending on whether the components are applied during the training phase or the deployment phase. In such scenarios, the training and deployment of the neural network model are performed by different parties. For example, the neural network training module 150 and training data store 170 applied during the training phase can be employed by a first party (e.g., to train a neural network model) and the neural network deployment module 160 applied during the deployment phase can be performed by a second party (e.g., to deploy the neural network model).

Referring to the image pre-processing module 145, it obtains images captured by the image generation system 120 and pre-processes the images. Generally, pre-processing the images enables more uniform and accurate analysis of the images by the neural network.

In various embodiments, the image pre-processing module 145 pre-processes an image by removing uninformative tiles or uninformative regions of one or more tiles of the image. For example, the image pre-processing module 145 may remove tiles or regions of tiles in which no tissue is present. As another example, the image pre-processing module 145 may remove tiles where less than a threshold area (e.g., less than 10%, less than 20%, less than 30%, less than 40%, or less than 50%) of the tile includes tissue. As another example, the image pre-processing module 145 may perform an image recognition to identify presence of pen marks on a tile of an image. Having identified a presence of pen marks, the image pre-processing module 145 can remove the regions of tiles that include the pen marks. The image pre-processing module 145 may remove uninformative regions of one or more tiles of training images during the training phase and/or may remove uninformative regions of one or more tiles of images during the deployment phase.

In various embodiments, the image pre-processing module 145 pre-processes an image by performing an image stain augmentation of the image. Generally, performing an image stain augmentation of an image includes generation of additional images. As one example, the image pre-processing module 145 performs an image stain augmentation of an image by modifying stain intensity or stain contrast of tiles of the image. As another example, the image pre-processing module 145 performs an image stain augmentation of an image by performing color jittering or color normalization of tiles of the image. As another example, the image pre-processing module 145 performs an image stain augmentation of an image by performing horizontal, vertical, or rotational flips of tiles of the image.

In various embodiments, image stain augmentation is only performed on training images (and not on images that are analyzed during the deployment phase), thereby enabling the generation of additional training images that can be used to train the neural network. In other words, the image pre-processing module 145 performs an image stain augmentation during the training phase to diversify and supplement the total training image set that is used to train the neural network. Thus, by training the neural network on the diversified training images, the neural network can handle tiles of varying stain intensities, varying stain colorations, and varying rotations and orientations.

The image pre-processing module 145 provides processed images to either the neural network training module 150 (during the training phase) or to the neural network deployment module 160 (during the deployment phase). In some embodiments, the image pre-processing module 145 stores the processed images in the training data store 170 such that those images can be later retrieved for training a neural network.

The neural network training module 150 trains neural network models using training data derived from training individuals. The neural network deployment module 160 implements neural network models to analyze individual tiles of images to generate a genotype prediction for the subject 110. Training machine learning models (e.g., neural network models) and deploying machine learning models (e.g., neural network models) are described in further detail below.

### Methods for Predicting Genotypes for Subjects

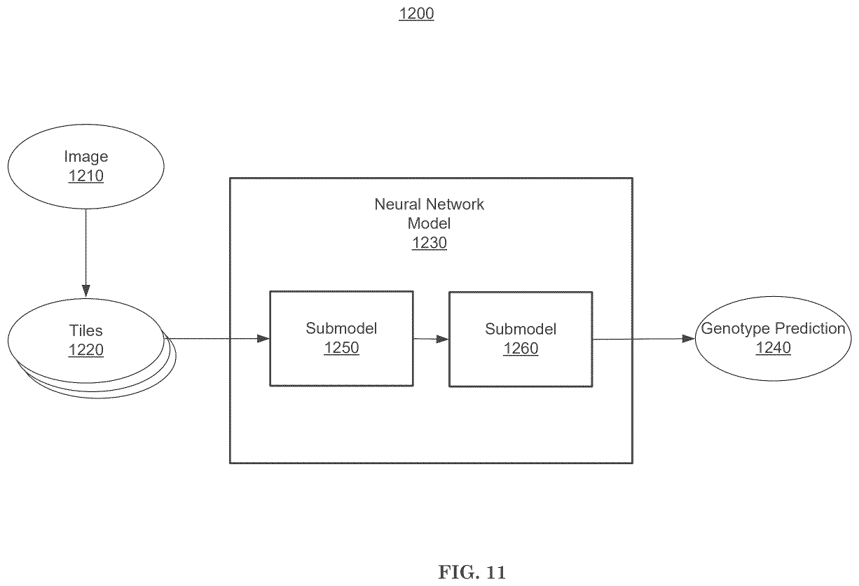

Embodiments described herein include methods for predicting a genotype for a subject by applying a trained neural network model. Such methods can be performed by the genotype prediction system 130 described in FIG. 10. Reference will further be made to FIG. 11 which depicts an example flow diagram 200 for determining a genotype prediction 1240 for a subject, in accordance with an embodiment.

The flow diagram 200 begins with an image 1210, such as an image captured by the image generation system 120. In various embodiments, the image 1210 previously undergoes image pre-processing, as is described above in context of the image pre-processing module 145 in FIG. 10. In such embodiments, image 1210 shown in FIG. 11 represents a pre-processed image.

A plurality of tiles 1220 is generated from the image 1210. Generally, each tile 1220 represents a subset of the image 1210. For example, if the image 1210 has dimensions of M pixels by N pixels, then a tile 1220 can have dimensions of M/X pixels by N/Y pixels, where the values of M/X and N/Y are constant values. In various embodiments, each tile 1220 has the same dimensions as every other tile 1220. In various embodiments, each tile 1220 has a dimension of 224×224 pixels.

The individual tiles 1220 are provided as input to the neural network model 1230. Generally, the neural network model 1230 analyzes the individual tiles 1220 and determines a prediction, such as the genotype prediction 1240. In various embodiments, the neural network model 1230 determines a prediction that is informative for determining the genotype prediction 1240. For example, the neural network model 1230 may determine a score that can then be translated to a genotype prediction 1240.

In various embodiments, the genotype prediction is determined by comparing the score to a threshold. If the score is above the threshold, the genotype prediction is a first classification. If the score is below the threshold, the genotype prediction is a second classification.

As shown in FIG. 11, the neural network model 1230 includes a first submodel 1250 and a second submodel 1260. In various embodiments, the neural network model 1230 only includes a first submodel 1250 and does not include a second submodel 1260. Thus, the genotype prediction 1240 is determined from the output of the first submodel 1250. In various embodiments, the first submodel 1250 analyzes the individual tiles 1220 and determines tile-level predictions or tile-level features informative for a tile-level prediction, but does not put out a tile-level prediction. For example, in one embodiments such as that shown in FIG. 11 that involve both a first submodel 1250 and a second submodel 1260, the first submodel 1250 outputs tile-level features that are then input into the second submodel. As another example, in embodiments in which the neural network model 1230 only includes a first submodel 1250, the first submodel outputs a tile-level prediction that is used to determine a genotype prediction 1240.

For example, if there are a total of Z tiles 1220 that are input into the neural network model 1230, the first submodel 1250 outputs Z different tile-level predictions or Z different tile-level features, each tile-level prediction or tile-level feature associated with a tile 1220. In various embodiments, each tile-level prediction can be a categorization of the tile. For example, a first categorization can be a wildtype status while a second categorization can be an altered genotype status. In various embodiments, each tile-level prediction can be a probability (e.g., between 0 and 1) reflecting the likely probability of a genotype status (e.g., wildtype or altered genotype status) of the tile. A tile-level feature can be a vector (e.g., an array of numbers) corresponding to the tile.

In various embodiments, the first submodel 1250 is a convolutional neural network comprised of multiple layers, multiple nodes per layer, and parameters associated with nodes of the layers. Thus, the convolutional neural network analyzes individual tiles by propagating the tiles through the multiple layers to generate the tile-level prediction (or tile-level feature).

In various embodiments, the neural network model 1230 only includes a first submodel 1250 and does not include a second submodel 1260. In such embodiments, the tile-level predictions (or tile-level feature) outputted by the first submodel 1250 can be analyzed to determine the genotype prediction 1240. For example, the tile corresponding to the highest tile probability can be selected and the highest tile probability is compared to a threshold value. If the highest tile probability is greater than the threshold value, the slide is labeled as a first classification. If the highest tile probability is less than the threshold value, the slide is labeled as a second classification. In various embodiments, the threshold value is 0.1, 0.2, 0.3, 0.4, 0.5, 0.6, 0.7, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99.

In various embodiments, such as the embodiment shown in FIG. 11, the neural network model 1230 includes both the first submodel 1250 and the second submodel 1260. Therefore, the individual tile-level predictions are provided as input into the second submodel 1260. In various embodiments, the first submodel 1250 generates tile-level features (e.g., features informative for aggregating the tiles) that are then provided as input to the second submodel 1260. Here, the second submodel 1260 generates an image-level prediction (also referred to herein as a “slide-level prediction”) by aggregating across the individual tile-level predictions or tile-level features.

In various embodiments, the second submodel 1260 is a neural network, hereafter referred to as an attention network. Thus, the attention network can be composed of multiple layers, multiple nodes per layer, and parameters associated with nodes of the layers. Generally, the attention network is trained to aggregate tile-level predictions or tile-level features to generate an image-level prediction. Thus, the attention network can learn to more heavily weigh certain tile-level predictions or tile-level features associated with tiles that are more informative for determining a genotype and conversely, to assign less weight to certain tile-level predictions or tile-level features associated with tiles that are less informative for determining a genotype.

In various embodiments, the second submodel 1260 outputs the genotype prediction 1240. In various embodiments, the second submodel 1260 outputs a score that is informative of the genotype prediction 1240. For example, the second submodel 1260 may output a constant value that is indicative of a classification of the genotype (e.g., wildtype or altered). As a specific example, a value of “0” can indicate a classification of a wildtype genotype whereas a value of “1” can indicate a classification of an altered genotype.

In various embodiments, the predicted genotype 140 for the subject can be displayed to a user e.g., a clinician user. Thus, the clinician user can inform the subject of the predicted genotype. In various embodiments, additional/other information can be displayed to a user e.g., a clinician user. For example, one or more tiles that were the most informative for generating the predicted genotype 140 can be displayed to the user. This enables the user to perform a manual check of the one or more tiles to ensure that no confounding image artifacts (e.g., pen marks or stain discoloration) led to the predicted genotype 140.

### Example Machine Learning Model for Predicting Genotypes

Embodiments disclosed herein involve training and deploying machine learning models for predicting a genotype a subject. Generally, a machine learning model is structured such that it analyzes individual tiles of an image and outputs an image-level prediction that is informative of the subject's genotype. In particular embodiments, the machine learning model is a neural network model (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, or deep bi-directional recurrent networks)). Although the description herein refers to neural network models, in other embodiments, the machine learning model is any one of a regression model (e.g., linear regression, logistic regression, or polynomial regression), decision tree, random forest, gradient boosted machine learning model, support vector machine, Naïve Bayes model, k-means cluster, or any combination thereof.

In various embodiments, the machine learning model includes two or more submodels. For example, the machine learning model can include two submodels, a first submodel that analyzes individual tiles and outputs tile-level predictions, as well as a second submodel that the tile-level predictions and outputs an image-level prediction. Thus, the output of the first submodel serves as input to the second submodel. In particular embodiments, both the first submodel and the second submodel are neural networks (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks), or any combination thereof. In particular embodiments, the first submodel is a convolutional neural network.

The machine learning model (and its submodels) can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the machine learning model is trained using supervised learning algorithms, unsupervised learning algorithms, semi-supervised learning algorithms (e.g., partial supervision), weak supervision, transfer, multi-task learning, or any combination thereof. In particular embodiments, the machine learning model is trained using weak supervision techniques. In particular embodiments, the machine learning model is trained using a deep learning algorithm.

In various embodiments, the machine learning model (and its submodels) has one or more parameters, such as hyperparameters or model parameters. Hyperparameters are generally established prior to training. In various embodiments, hyperparameter optimization (e.g., grid search) is performed via cross validation. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, node values in a decision tree, and coefficients in a regression model. The model parameters of the machine learning model are trained (e.g., adjusted) using the training data to improve the predictive capacity of the machine learning model.

Referring again to FIG. 10, the neural network training module 150 trains the machine learning model using training data. In various embodiments, the training data can be obtained and/or derived from a publicly available database. For example, the training data can be obtained and/or derived from the National Cancer Institute GDC Data portal and/or the cBioPortal for Cancer Genomics. In some embodiments, the training data can be obtained and collected independent of publicly available databases e.g., by capturing images from a plurality of training individuals. Such training data can be a custom dataset.

In various embodiments, the training data can be stored and/or retrieved from training data store 170. Generally, the training data includes training images of H&E stained tissue slides derived from training individuals (e.g., individuals with a known wildtype or altered genotype). As described above, the training images may have undergone pre-processing. For example, the training images may have undergone a quality control process to remove image artifacts such as pen marks. Additionally, training images may have been generated via stain-based augmentation, which diversifies and increases the number of training images that can be used for training. For example, a single image can be pre-processed using a stain-based augmentation process to generate additional images of different stain colorations. As a specific example, a single training image can undergo the stain-based augmentation process to generate ten training images of different stain colorations. This prevents different stain colorations from influencing the predictions generated by the machine learning model.

In various embodiments, the training data can be obtained from a split of a dataset. For example, the dataset can undergo a 50:50 training:testing dataset split. In some embodiments, the dataset can undergo a 60:40 training:testing dataset split. In some embodiments, the dataset can undergo a 80:20 training:testing dataset split. In some embodiments, the dataset can undergo a 70:15:15 training:testing:validation dataset split.

In various embodiments, the training data used for training the machine learning model includes reference ground truths that indicate the genotype status of the training individual. In various embodiments, the reference ground truths in the training data are binary values, such as “1” or “0.” For example, a training individual that has a known wildtype genotype can be identified in the training data with a value of “0” whereas a training individual that has a known altered genotype can be identified in the training data with a value of “1.”

In various embodiments, the neural network training module 150 trains the machine learning model using the training data to minimize a loss function such that the machine learning model can better predict the genotype status based on the input (e.g., tiles of the training image). Here, the neural network training module 150 may backpropagate a loss value and adjust the parameters of the machine learning model to minimize the loss value. In various embodiments, the loss function is constructed for any of a least absolute shrinkage and selection operator (LASSO) regression, Ridge regression, or ElasticNet regression. In particular embodiments, the loss function is the cross-entropy loss between the predicted label versus the true label (e.g., label can be a binary label of true or false for FGFR in bladder cancer). As described above, in some embodiments, the machine learning model is a neural network model with at least a first submodel (e.g., a convolutional neural network) and a second submodel (e.g., an attention network). In various embodiments, the first submodel and the second submodel are jointly trained. Therefore, the neural network training module 150 backpropagates the loss value and trains the neural network model by jointly adjusting the parameters of both the first submodel and the parameters of the second submodel. In other embodiments, the first submodel and the second submodel are separately trained. Thus, the neural network training module 150 trains the neural network model to minimize a loss function by separately adjusting the parameters of the first submodel and the parameters of the second submodel.

In various embodiments, machine learning models disclosed herein achieve a performance metric. Example performance metrics include an area under the receiver operating curve (auROC) of a receiver operating curve, a positive predictive value, and/or a negative predictive value. In various embodiments, machine learning models disclosed herein exhibit an auROC value of at least 0.5. In various embodiments, machine learning models disclosed herein exhibit an auROC value of at least 0.6. In various embodiments, machine learning models disclosed herein exhibit an auROC value of at least 0.7. In various embodiments, machine learning models disclosed herein exhibit an auROC value of at least 0.8. In various embodiments, machine learning models disclosed herein exhibit an auROC value of at least 0.9. In various embodiments, machine learning models disclosed herein exhibit an auROC value of at least 0.95. In various embodiments, machine learning models disclosed herein exhibit an auROC value of at least 0.99. In various embodiments, machine learning models disclosed herein exhibit an auROC value of at least 0.51, at least 0.52, at least 0.53, at least 0.54, at least 0.55, at least 0.56, at least 0.57, at least 0.58, at least 0.59, at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, at least 0.74, at least 0.75, at least 0.76, at least 0.77, at least 0.78, at least 0.79, at least 0.80, at least 0.81, at least 0.82, at least 0.83, at least 0.84, at least 0.85, at least 0.86, at least 0.87, at least 0.88, at least 0.89, at least 0.90, at least 0.91, at least 0.92, at least 0.93, at least 0.94, at least 0.95, at least 0.96, at least 0.97, at least 0.98, or at least 0.99.

### Example Method for Predicting Genotypes for Subjects

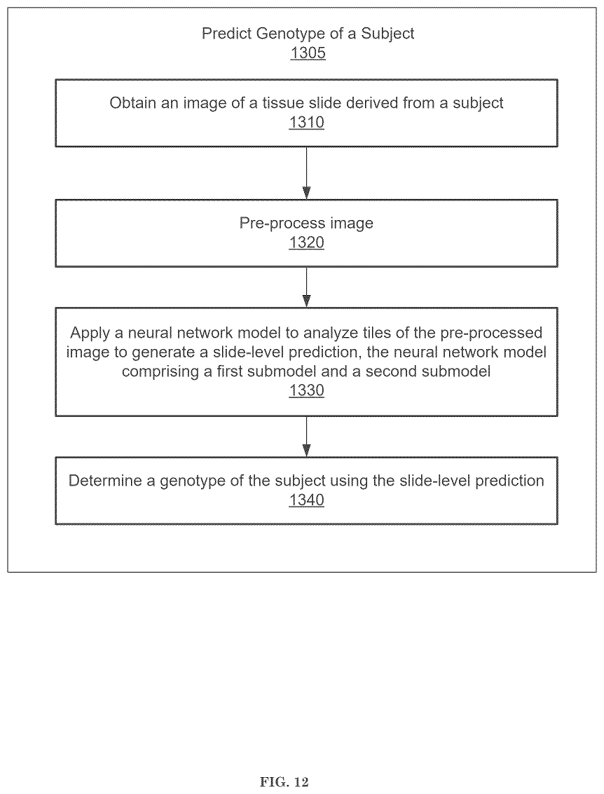

FIG. 12 is an example flow process for determining a genotype prediction for a subject, in accordance with a first embodiment. As shown in FIG. 12, the flow process 1305 includes steps 1310, 1320, 1330, and 1340.

Step 1310 involves obtaining an image of a tissue slide derived from a subject. In various embodiments, the tissue slide is a H&E stained biopsy slide. In various embodiments, the tissue is a biopsy of the bladder or prostate of the subject.

Step 1320 involves pre-processing the image using a quality control process. In various embodiments, pre-processing the image involves improving the quality of the image e.g., by removing uninformative regions of the image and/or augmenting the tissue stain. Thus, a pre-processed image allows the image to be more readily manageable such that the neural network can appropriately analyze the image with limited impact from confounding factors (e.g., from image artifacts).

Step 1330 involves applying a neural network model to analyze tiles of the pre-processed image. In various embodiments, the neural network model generates a slide-level prediction. In various embodiments, the slide-level prediction includes a score. In various embodiments, the neural network model generates tile-level predictions based on the analysis of the individual tiles, and subsequently aggregates the tile-level predictions to generate the slide-level prediction. In various embodiments, the neural network model includes at least a first submodel (e.g., a convolutional neural network) and a second submodel (e.g., an attention network for performing tile aggregation to generate the slide-level prediction).

Step 1340 involves determining a genotype for the subject according to the slide-level prediction. For example, the slide-level prediction may include a score and therefore, the subject's genotype is classified according to the score. In various embodiments, the subject's genotype is classified by comparing the score to a threshold. If the score is above the threshold, the subject's genotype is classified in a first classification. If the score is below the threshold, the subject's genotype is classified in a second classification.

In various embodiments, the subject undergoes additional molecular testing based on the slide-level prediction. For example, if the slide-level prediction indicates that the subject likely exhibits an altered genotype, the subject undergoes additional molecular testing to confirm the in silico results. Generally, this enables the prioritization of subjects for molecular testing, as subjects who are predicted to exhibit an altered genotype status can be prioritized ahead of subjects who are predicted to exhibit a wildtype genotype status.

In various embodiments, once confirmed through the molecular testing, appropriate medical treatment or medical advice can be provided to subjects based on the altered genotype status. In various embodiments, a guided intervention can be selected for the subject based on the subject's genotype. For example, if the predicted genotype indicates presence of a mutated target (e.g., such as a mutated fibroblast growth factor receptor (FGFR)), then a therapeutic intervention (e.g., BALVERSA) can be selected for the subject. In some embodiments, the subject can be enrolled in a clinical trial to receive a therapeutic intervention, given that the predicted genotype indicates presence of a mutated target.

### Cancers and Mutations

Methods described herein involve implementing neural network models for predicting cancer relevant genotypes of subjects. In various embodiments, the cancer in the subject can include one or more of: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, stomach cancer, thyroid cancer, head and neck carcinoma, large bowel cancer, hematopoietic cancer, testicular cancer, colon and/or rectal cancer, uterine cancer, or prostatic cancer. In some embodiments, the cancer in the subject can be a metastatic cancer, including any one of bladder cancer, breast cancer, colon cancer, kidney cancer, lung cancer, melanoma, ovarian cancer, pancreatic cancer, prostatic cancer, rectal cancer, stomach cancer, thyroid cancer, or uterine cancer. In particular embodiments, the cancer is bladder cancer. In particular embodiments, the cancer is prostate cancer. In particular embodiments, the cancer is lung cancer.

In various embodiments, methods disclosed herein involve predicting a genotype relevant for bladder cancer, such as a fibroblast growth factor receptor (FGFR). In such embodiments, a guided therapy can be provided to the subject based on the FGFR status of the subject. FGFR status can refer to a wild type FGFR or a FGFR alteration. In various embodiments, a FGFR alteration refers to any of a FGFR3 mutation, a FGFR3 fusion, or a combination of a FGFR3 mutation and FGFR3 fusion. In various embodiments, a FGFR alteration refers to any of a FGFR2 mutation, a FGFR2 fusion, or a combination of a FGFR2 mutation and FGFR2 fusion.

Example FGFR3 point mutations are described below in Table 1.

TABLE 1
therascreen FGFR RGQ RT-PCR Kit
assays targets: Point mutations

| | AMINO | | | |
|---|---|---|---|---|
| | ACID | CDS | | |
| GENE | VARIANT | MUTATION | COSMIC ID | EXONS |
| FGFR3 | p.R248C | c.742C > T | COSM714 | 7 |
| FGFR3 | p.G370C | c.1108G > T | COSM716 | 10 |
| FGFR3 | p.S249C | c.746C > G | COSM715 | 7 |
| FGFR3 | p.Y373C | c.1118A > G | COSM718 | 10 |

Example FGFR3 fusions are described below in Table 2.

TABLE 2
therascreen FGFR RGQ RT-PCR Kit assay targets: Fusions

| | GENES | GENOMIC | |
|---|---|---|---|
| FUSION ID | INVOLVED | BREAKPOINT | EXONS |
| FGFR3:TACC3V1 | FGFR3 | chr4: 1808661 C | 17 |
| | TACC3 | G chr4: 1741428 | 11 |
| FGFR3:TACC3V3 | FGFR3 | chr4: 1808661 C | 17 |
| | TACC3 | G chr4: 1739324 | 10 |
| FGFR3:BAIAP2L1 | FGFR3 | chr4: 1808661 C | 17 |
| | BAIAP2L1* | A chr7: 97991744 | 2 |
| FGFR2:BICC1 | FGFR2 | chr10: 123243211 G | 17 |
| | BICC1* | A chr10: 60461834 | 3 |
| FGFR2:CASP7 | FGFR2 | chr10: 123243211 G | 17 |
| | CASP7* | A chr10: 115457252 | 2 |

In various embodiments, methods disclosed herein involve predicting a genotype relevant for prostate cancer, such as BRCA1 and/or BRCA2 genes. For example, genotypes relevant for prostate cancer can include single nucleotide polymorphisms (SNPs), copy number variations (CNVs), or gene fusions involving BRCA1 or BRCA2 genes.

Embodiments described herein involve implementing machine learning models (e.g., neural network models) for predicting genotypes for subjects. In various embodiments, an intervention is selected for the subject based on the predicted genotype. In various embodiments, the intervention can be any one of: application of a diagnostic, application of a prophylactic therapeutic agent, or a subsequent action. Example subsequent actions can include a subsequent testing of the subject or a test sample from the subject to confirm the in silico genotype prediction.

In particular embodiments, if the predicted genotype of the subject is an altered genotype, the test sample from the subject can be provided for additional molecular test screening to confirm that the subject exhibits an altered genotype. In such embodiments, if the additional molecular test screening confirms that the subject exhibits an altered genotype, the subject can be deemed eligible for enrollment in a clinical trial. In particular embodiments, if the predicted genotype of the subject is a wildtype genotype, the subject can be excluded from subsequent analysis for potential enrollment in a clinical trial. In such embodiments, the in silico process serves as a prospective screen that eliminates a number of subjects that likely exhibit a wildtype genotype, and therefore, should not be enrolled in the clinical trial. This saves having to conduct molecular screens for every single patient, which is timely and costly.

In various embodiments, an intervention comprising BALVERSA (erdafitinib) can be selected for a subject if the predicted genotype of the subject indicates a presence of a mutated FGFR. As another example, an intervention comprising a Poly (ADP-ribose) polymerase (PARP) inhibitor and specifically, ZEJULA (Niraparib), can be selected for a subject if the predicted genotype of the subject indicates a presence of a an altered BRCA gene (e.g., altered BRCA1 or BRCA2).

In various embodiments, a therapeutic agent can be selected and/or administered to the subject based on the predicted genotype for the subject. The selected therapeutic agent is likely to delay or prevent the development of the cancer, such as prostate cancer or bladder cancer. Exemplary therapeutic agents include chemotherapies, energy therapies (e.g., external beam, microwave, radiofrequency ablation, brachytherapy, electroporation, cryoablation, photothermal ablation, laser therapy, photodynamic therapy, electrocauterization, chemoemboilization, high intensity focused ultrasound, low intensity focused ultrasound), antigen-specific monoclonal antibodies, anti-inflammatoires, oncolytic viral therapies, or immunotherapies. In various embodiments, the selected therapeutic agent is an energy therapy and the amount (e.g., dose and duration) of the energy applied can be tailored to achieve a desired therapeutic effect. In various embodiments the therapeutic agent is a small molecule or biologic, e.g. a cytokine, antibody, soluble cytokine receptor, anti-sense oligonucleotide, siRNA, etc. Such biologic agents encompass muteins and derivatives of the biological agent, which derivatives can include, for example, fusion proteins, PEGylated derivatives, cholesterol conjugated derivatives, and the like as known in the art. Also included are antagonists of cytokines and cytokine receptors, e.g. traps and monoclonal antagonists. Also included are biosimilar or bioequivalent drugs to the active agents set forth herein.

Therapeutic agents for bladder cancer can include therapeutics such as BALVERSA (erdafitinib), Atezolizumab, Avelumab, BAVENCIO (Avelumab), Cisplatin, Doxorubicin Hydrochloride, Enfortumab Vedotin-ejfv, erdafitinib, JELMYTO (Mitomycin), KEYTRUDA (Pembrolizumab), Nivolumab, OPDIVO (Nivolumab), PADCEV (Enfortumab Vedotin-ejfv), Pembrolizumab, Sacituzumab Govitecan-hziy, TECENTRIQ (Atezolizumab), TEPADINA (Thiotepa), Thiotepa, TRODELVY (Sacituzumab Govitecan-hziy, Valrubicin, and VALSTAR (Valrubicin). In particular embodiments, the therapeutic is BALVERSA (erdafinitib).

Therapeutic agents for prostate cancer can include a Poly (ADP-ribose) polymerase (PARP) inhibitor, Abiraterone Acetate, Apalutamide, Bicalutamide, Cabazitaxel, CASODEX (Bicalutamide), Darolutamide, Degarelix, Docetaxel, ELIGARD (Leuprolide Acetate), Enzalutamide, ERLEADA (Apalutamide) FIRMAGON (Degarelix), Flutamide, Goserelin Acetate, JEVTANA (Cabazitaxel), Leuprolide Acetate, LUPRON DEPOT (Leuprolide Acetate) LYNPARZA (Olaparib), Mitoxantrone Hydrocholoride, NILANDRON (Nilutamide), Nilutamide, NUBEQA (Darolutamide), Olaparib, ORGOVYX (Relugolix), PROVENGE (Sipuleucel-T), Radium 223 Dichloride, Relugolix, RUBRACA (Rucaparib Camsylate), Rucaparib Camsylate, Sipuleucel-T, TAXOTERE (Docetaxel), XOFIGO (Radium 223 Dichloride), XTANDI (Enzalutamide), YONSA (Abiraterone Acetate), ZOLADEX (Goserelin Acetate), and ZYTIGA (Abiraterone Acetate). In particular embodiments, a therapeutic agent for prostate cancer is a PARP inhibitor and specifically, ZEJULA (Niraparib). In particular embodiments, a therapeutic agent for lung cancer is Rybervant (Amivantamab).

In various embodiments, one or more of the therapeutic agents described can be combined as a combination therapy for treating the subject.

In various embodiments, a pharmaceutical composition can be selected and/or administered to the subject based on at least the predicted genotype for the subject, the selected therapeutic agent likely to exhibit efficacy against the cancer. In various embodiments, a pharmaceutical composition can be selected and/or administered to the subject based on a molecular test that is conducted based on the prediction that the subject's tumor exhibits a mutated genotype. For example, if the predicted genotype indicates a mutated genotype for the subject's tumor and the subsequent molecular test confirms the mutated genotype of the subject's tumor, the pharmaceutical composition can be selected and/or administered to the subject. A pharmaceutical composition administered to an individual includes an active agent such as the therapeutic agent described above. The active ingredient is present in a therapeutically effective amount, i.e., an amount sufficient when administered to treat a disease or medical condition mediated thereby. The compositions can also include various other agents to enhance delivery and efficacy, e.g. to enhance delivery and stability of the active ingredients. Thus, for example, the compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant.

The pharmaceutical compositions or therapeutic agents described herein can be administered in a variety of different ways. Examples include administering a composition containing a pharmaceutically acceptable carrier via oral, intranasal, intramodular, intralesional, rectal, topical, intraperitoneal, intravenous, intramuscular, subcutaneous, subdermal, transdermal, intrathecal, endobronchial, transthoracic, or intracranial method.

### Computer Implementation

The methods of the invention, including the methods of implementing neural network models for predicting genotypes of subjects, are, in some embodiments, performed on one or more computers.

For example, the building and deployment of a neural network model can be implemented in hardware or software, or a combination of both. In one embodiment of the invention, a machine-readable storage medium is provided, the medium comprising a data storage material encoded with machine readable data which, when using a machine programmed with instructions for using said data, is capable of executing the training or deployment of neural network models and/or displaying any of the datasets or results described herein. The invention can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high-level procedural or object-oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In some embodiments, the methods of the invention, including the methods for predicting genotypes of subjects involving implementing neural network models, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 13 illustrates an example computer for implementing the entities shown in FIGS. 9, 10, 11, and 12. The computer 1400 includes at least one processor 1402 coupled to a chipset 1404. The chipset 1404 includes a memory controller hub 1420 and an input/output (I/O) controller hub 1422. A memory 1406 and a graphics adapter 1412 are coupled to the memory controller hub 1420, and a display 1418 is coupled to the graphics adapter 1412. A storage device 1408, an input device 1414, and network adapter 1416 are coupled to the I/O controller hub 1422. Other embodiments of the computer 1400 have different architectures.

The storage device 1408 is a non-transitory computer-readable storage medium such as a hard drive, compact disk read-only memory (CD-ROM), DVD, or a solid-state memory device. The memory 1406 holds instructions and data used by the processor 1402. The input interface 1414 is a touch-screen interface, a mouse, track ball, or other type of pointing device, a keyboard, or some combination thereof, and is used to input data into the computer 1400. In some embodiments, the computer 1400 may be configured to receive input (e.g., commands) from the input interface 1414 via gestures from the user. The network adapter 1416 couples the computer 1400 to one or more computer networks.

The graphics adapter 1412 displays images and other information on the display 1418. In various embodiments, the display 1418 is configured such that the user may (e.g., radiologist, oncologist, pulmonologist) may input user selections on the display 1418 to, for example, predict a genotype for a patient or order any additional exams or procedures. In one embodiment, the display 1418 may include a touch interface. In various embodiments, the display 1418 can show one or more predicted genotypes for a subject. Thus, a user who accesses the display 1418 can inform the subject of the predicted genotypes for the subject. In various embodiments, the display 1418 can show information such as individual tiles of images that most heavily contributed to the predicted genotype for the subject. For example, such information can be useful for verifying that the predicted genotype was due to particular attributes in the tissue as opposed to image artifacts.

The computer 1400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 1408, loaded into the memory 1406, and executed by the processor 1402.

The types of computers 1400 used by the entities of FIG. 9 or 10 can vary depending upon the embodiment and the processing power required by the entity. For example, the genotype prediction system 130 can run in a single computer 1400 or multiple computers 1400 communicating with each other through a network such as in a server farm. The computers 1400 can lack some of the components described above, such as graphics adapters 1412, and displays 1418.

### Systems

Further disclosed herein are systems for implementing neural network models for predicting genotypes for subjects. In various embodiments, such a system can include at least the genotype prediction system 130 described above in FIG. 9. In various embodiments, the genotype prediction system 130 is embodied as a computer system, such as a computer system with example computer 1400 described in FIG. 13.

In various embodiments, the system includes an imaging device, such as an imaging generation system 120 described above in FIG. 9. In various embodiments, the system includes both the genotype prediction system 130 (e.g., a computer system) and an imaging generation system 120. In such embodiments, the genotype prediction system 130 can be communicatively coupled with the image generation system 120 to receive images captured from a subject. Thus, the genotype prediction system 130 implements, in silico, neural network models to analyze the images and to determine predictions of genotypes for the subject.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used, but some experimental error and deviation should be allowed for.

### Example 1: First Implementation of a Neural Network Model and Corresponding Issues

The goal was to develop and implement a machine learning model for predicting patient mutations, thereby enabling the pre-screening of patients for eligibility in clinical trials. By pre-screening patients using in silico methods, this would reduce the number of patients that are required to undergo molecular screening. Furthermore, in silico methods can be rapid and cost effective, which would encourage physicians to send patients for screening that have a high predicted likelihood of having a qualifying mutation. Altogether, an inexpensive mutation screening tool can lower the barrier to testing in real-world clinical setting and help match patients to the right therapy.

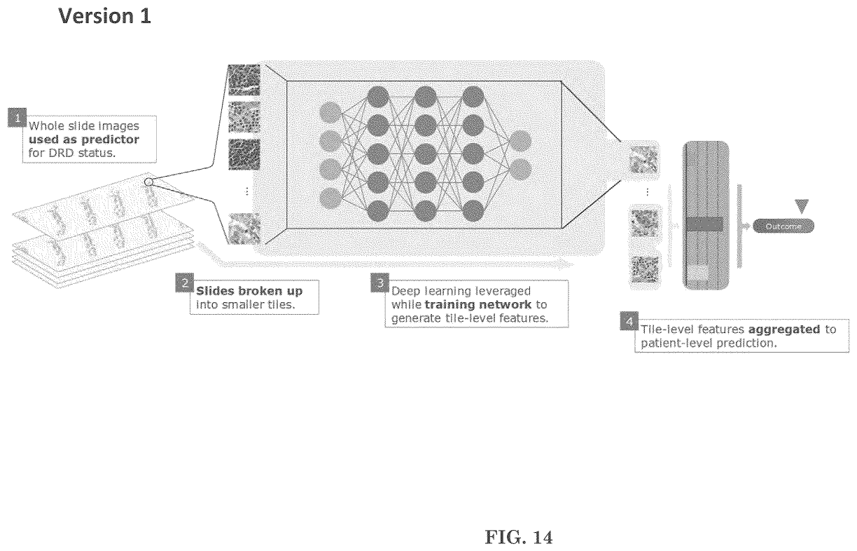

FIG. 14 depicts a first example process for generating a patient-level genotype prediction using slide images. Here, whole slide H&E images are analyzed. The slides were broken up into individual tiles which were then analyzed using a neural network to perform tile-level predictions. These tile-level predictions were aggregated to patient-level prediction. Here, the aggregation of tile-level predictions is performed by selecting the highest tile-level prediction in the slide (i.e., max. aggregation). Further description of this first example process is disclosed in Campanella, G., et al. Clinical-grade computational pathology using weakly supervised deep learning on whole slide images. Nat Med 25, 1301-1309 (2019), which is hereby incorporated by reference in its entirety.

However, two main issues arose in this analysis:
Slide quality (i.e., blurs, pen marks, dust/dirt)
Batch differences/variability

These issues are described in further detail below in Example 2.

### Example 2: Second Implementations of a Neural Network Model Incorporating Pre-Processing of Images

To address the modeling challenges described above and increase model performance, a second implementation of a neural network model was developed to include a quality control step (e.g., slide quality challenges) and a data augmentation step (e.g., batch differences).

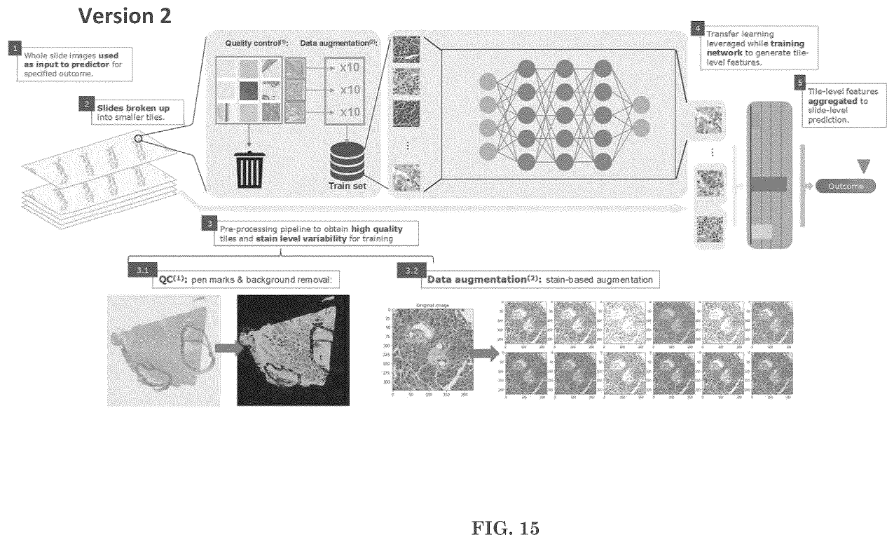

FIG. 15 depicts a second example process for generating a patient-level genotype prediction using slide images. Here, whole slide images were broken up into smaller tiles. Then, a quality control step and data augmentation step were performed on individual tiles. The quality control step removed pen marks and background. Pen marks and background intensity are image artifacts that can negatively affect the performance of a neural network that is analyzing the tiles. The data augmentation step involved a stain-based augmentation that increases and diversifies the number of differently stained images in the training set. This ensured that the trained neural network was trained on tiles of different stain intensities and colorations and therefore, predictions by the neural network were less influenced by stain variations across different tiles and samples.

The pre-processed tiles were provided as input to the neural network, which generated tile-level predictions that are each indicative of whether a particular tile likely corresponds to an altered status or wildtype status. The tile-level predictions were aggregated to generate a slide-level prediction. Here, the aggregation of tile-level predictions is performed by selecting the highest tile-level prediction in the slide (i.e., max. aggregation).

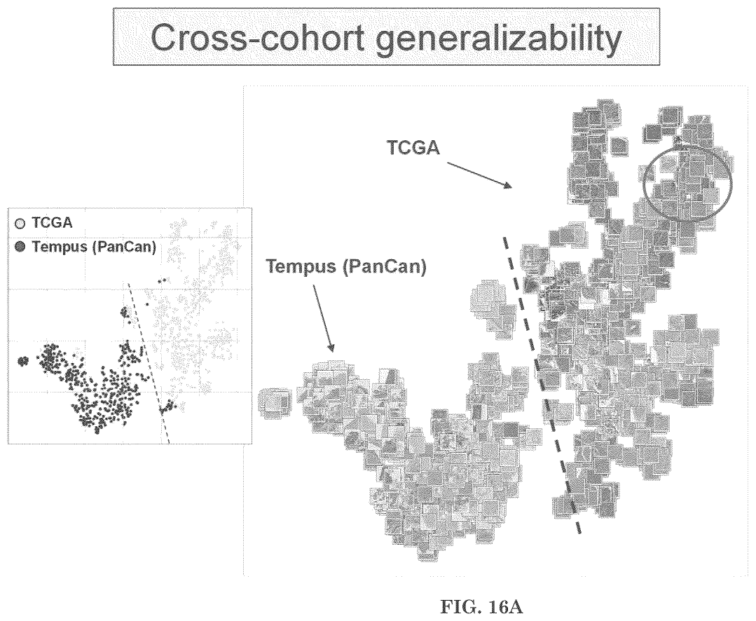

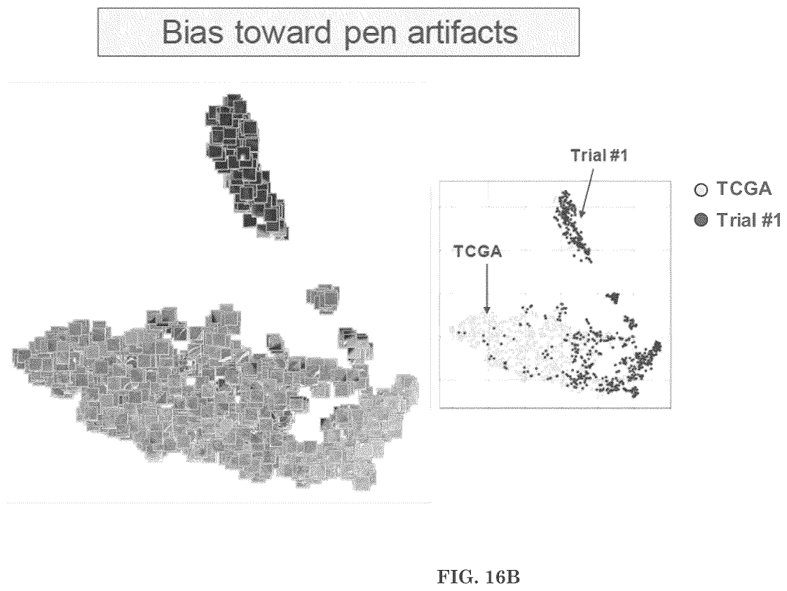

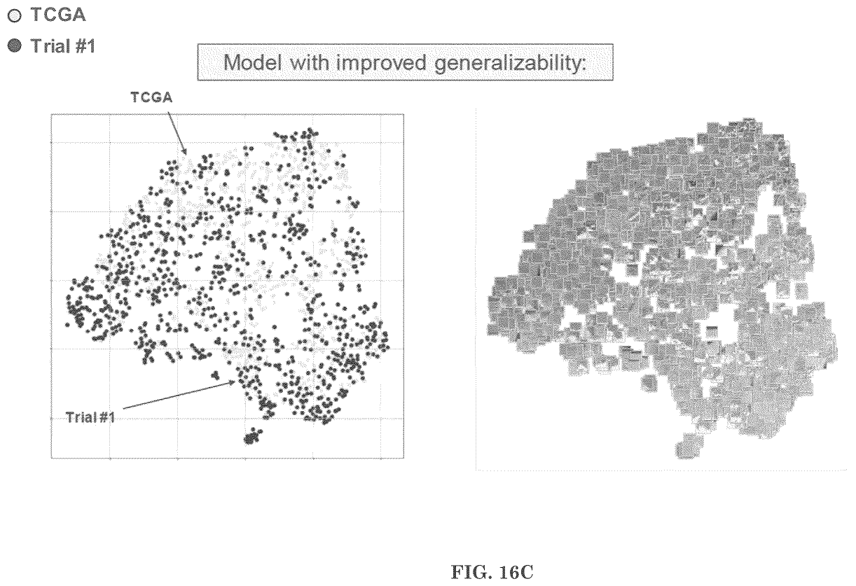

In general, the neural network's performance was improved when incorporating the pre-processing steps (e.g., quality control and data augmentation steps). First, FIG. 16A shows lack of cross-cohort generalizability as TCGA and Tempus samples are distinguished. To verify that the improvement was at least partially due to the reduction in bias towards pen artifacts, the neural network was analyzed when the pre-processing steps were and were not implemented. FIG. 16B shows the neural network's bias towards pen artifacts when a pre-processing step was not implemented. Notably, the neural network treated the TCGA samples significantly differently than the Trial #1 samples (e.g., as shown by their different cluster locations) due to the differences in pen marks on the images. This indicates that the presence of pen marks negatively influenced the prediction of the neural network. Conversely, FIG. 16C shows the neural network's improved treatment of TCGA and Trial #1 samples when the pre-processing steps were implemented. Notably, FIG. 16C shows that the neural network treated TCGA and Trial #1 samples in an unbiased manner as the respective tiles are indistinguishably clustered together. This indicates that the presence of pen marks did not influence the prediction of the neural network.

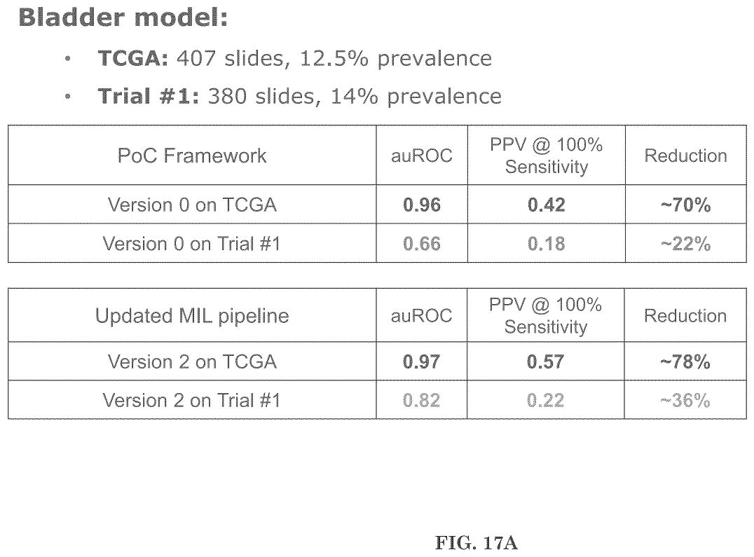

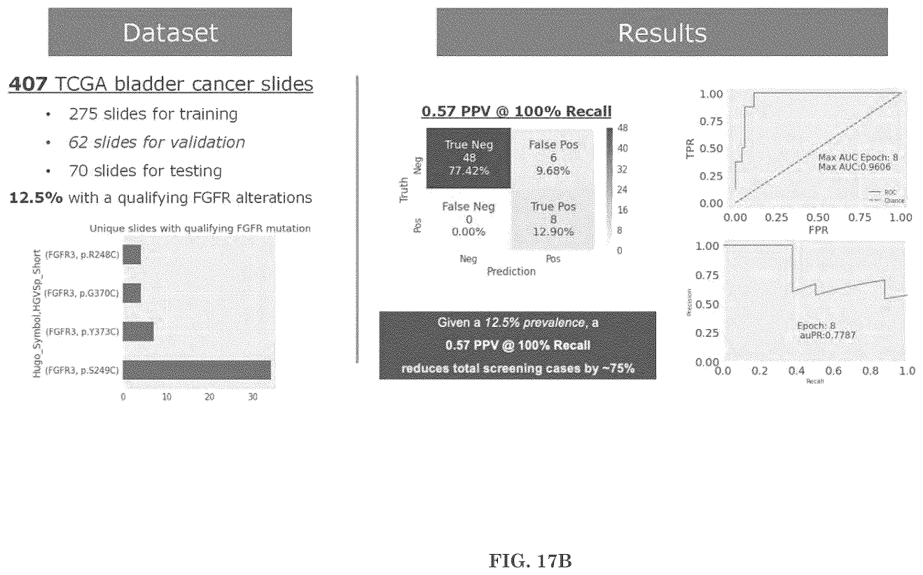

FIG. 17A depicts the performance of a model deployed in the second example process (described in FIG. 15) in comparison to a model deployed in the first example process (described in FIG. 14). Specifically, “Version 1” in FIG. 17A refers to the first example process described in FIG. 14 and “Version 2” in FIG. 17A refers to the second example process described in FIG. 15.

Generally, the incorporation of the pre-processing steps in “Version 2” e.g., the second example process (described in FIG. 15) improved the performance of the neural network when analyzing samples from both TCGA and Trial #1. As shown in FIG. 17A, each of the performance metrics (e.g., area under the receiver operating curve (auROC) and positive predictive value (PPV)) were improved when using “Version 2”.

FIG. 17B describes performance of a model tested on TCGA bladder cancer slides according to the second example process (described in FIG. 15). Here, the TCGA samples included 407 total TCGA bladder cancer slides. 275 slides were used for training, 62 slides were used for validation, and 70 slides were used for testing. A known 12.5% of the samples had qualifying fibroblast growth factor receptor (FGFR) alterations (e.g., mutations, fusions, or both). The results shown in FIG. 17B emphasize that the model deployed in accordance with the second example process described in FIG. 15 was able to achieve a PPV of 0.57 at 100% recall, which would reduce total screening cases by ˜75%. Thus, this indicates that the inclusion of the pre-processing steps significantly improved the performance of the model.

### Example 3: Third Implementation of a Neural Network Model in which Aggregation of Tile-Level Features is Learned

To further improve the performance of the neural network model in comparison to the models described in Examples 1 and 2, the neural network model was further constructed with an attention network such that an aggregation of tile-level features (e.g., normalized weighting values for each tile's feature vector used to summarize all the tiles of a particular image) is learned. Such a neural network model can achieve more reliable and informative probability scores, and can be more robust to image artifacts. Furthermore, having a trainable aggregation step was desirable so that the model would automatically learn how to aggregate the multiple tiles from the data to give accurate predictions.

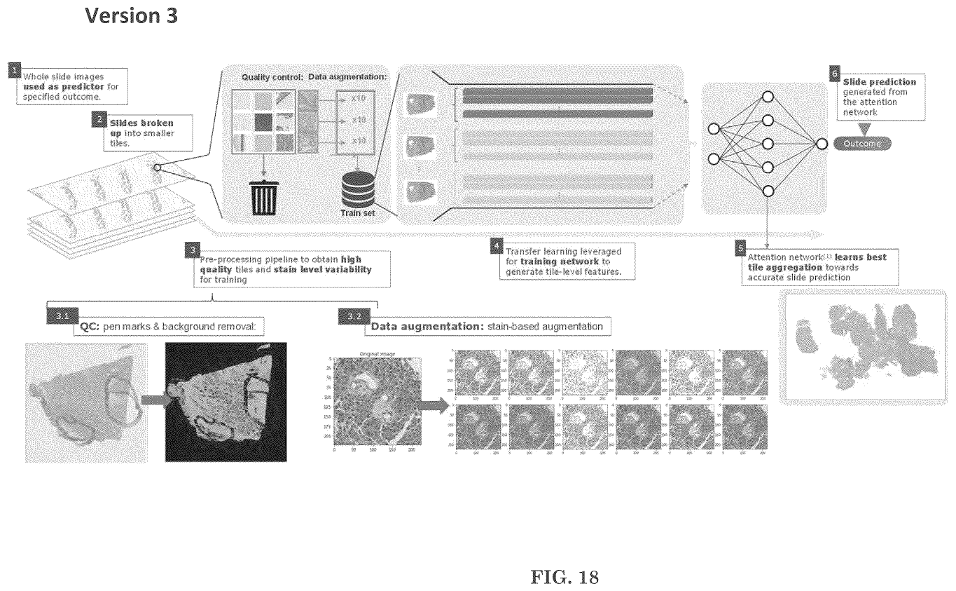

FIG. 18 depicts a third example process for generating a patient-level genotype prediction using slide images. Here, the third example process involves the implementation of an attention network, which learned the manner in which to aggregate the tile-level features to generate the slide-level prediction. As shown in FIG. 18, the third implementation further includes the pre-processing steps.

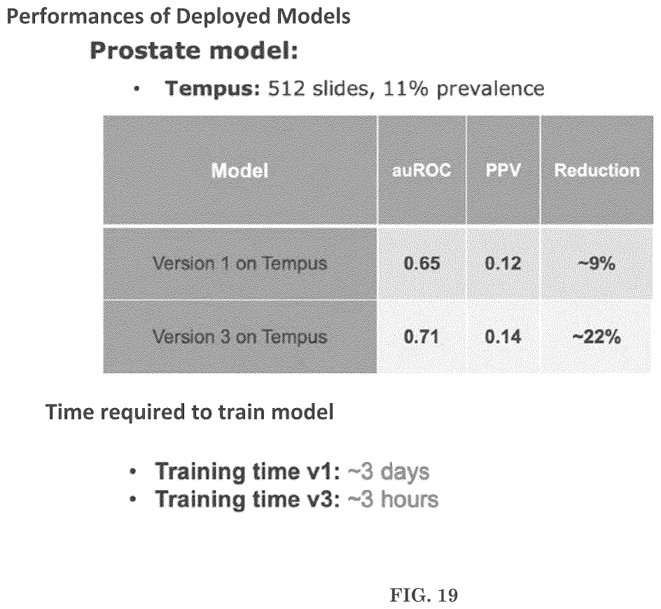

FIG. 19 depicts the performances of a model deployed in the third example process (described in FIG. 18) in comparison to a model deployed in the first example process (described in FIG. 14). Here, the models were tested on the Tempus dataset which includes 512 tissue slides with a known 11% prevalence rate of known alterations. Additionally, “Version 1” in FIG. 19 refers to the first example process described in FIG. 14 whereas “Version 3” in FIG. 19 refers to the third example process described in FIG. 18.

First, the model of the third example process was able to achieve improved performance in a significantly reduced training time. Namely, the “Version 1” model required 3 days of training time whereas the “Version 3” model required only 3 hours of training. Here, a pretrained CNN can be used for Version 3 and therefore, the focus of the training for the Version 3 model is on the attention network. In contrast for version 1, the CNN is trained using a fixed/non-learnable aggregation such as a max aggregation approach. Training the CNN (as required in Version 1 model) takes much longer time than training an attention model (as is required in Version 3 model) because the CNN includes many more parameters (i.e., looks at 2D image of each tile instead of probabilities/features per tile). Additionally, even with the reduced training time, the “Version 3” model achieved a higher performance metrics (e.g., auROC value=0.71 and PPV=0.14) in comparison to the “Version 1” model (auROC=0.65 and PPV=0.12).

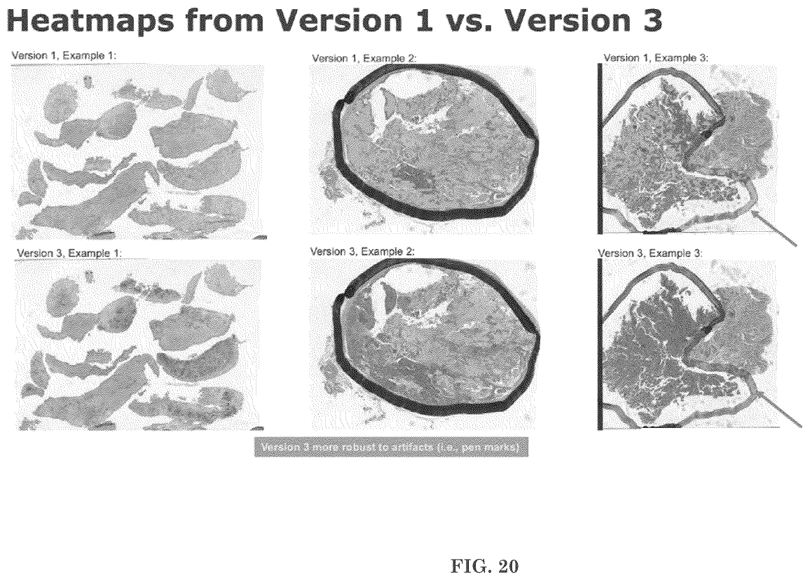

Furthermore, given that the “Version 3” model also incorporated the pre-processing steps, the “Version 3” model was also more robust to image artifacts, such as pen marks, in comparison to the “Version 1” model. Specifically, FIG. 20 shows respective heatmaps of the first example process (described in FIG. 14) and third example process (described in FIG. 8), which demonstrates that the third example process was more robust to image artifacts, such as pen marks.

Altogether, these results indicate that the third example predicts subject genotypes with reduced training and appropriate performance.

### Example 4: Fourth Implementation of a Neural Network Model in which First and Second Submodels are Jointly Trained

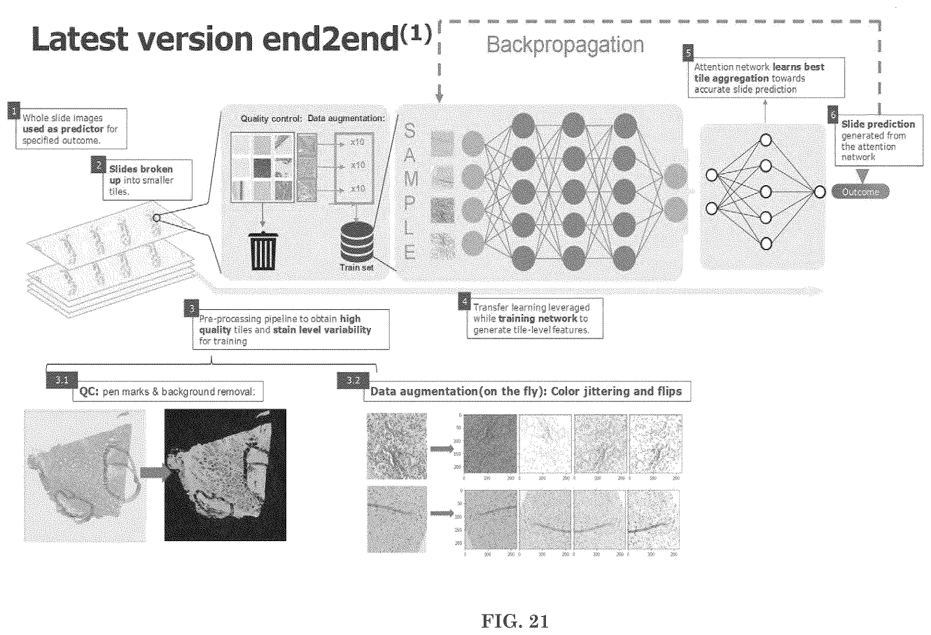

FIG. 21 depicts a fourth example process for generating a patient-level genotype prediction using slide images. Here, the fourth example process shown in FIG. 21 is an “End2End” model meaning that both the first submodel (e.g., neural network which generates tile-level features) and the second submodel (e.g., attention network which learns the best aggregation of tile-level features) are jointly trained. As shown in FIG. 21, the slide-level prediction is analyzed for its accuracy (e.g., as measured by a difference between ground truth and the slide-level prediction) and the value is backpropagated to jointly train the parameters of the neural network and the attention network.

The “End2End” version improves on Version 3 (described in Example 3) by jointly training the convolutional neural network with the attention module. A single backpropagation loop updates the weights of the attention as well as the CNN which enables the CNN to learn histopathology-based features.

The “End2End” version also performs stain augmentation on the fly using color jittering along with flipping/rotations, hence not requiring the need to do that manually beforehand. The memory constraint due to the large size of training patches (tiles) per slide is countered by randomly sampling ‘N’ number of patches (tiles) per slide in each batch and hence making the network fit in GPU memory.

### Example 5: Example Deployment of Pipeline Workflow

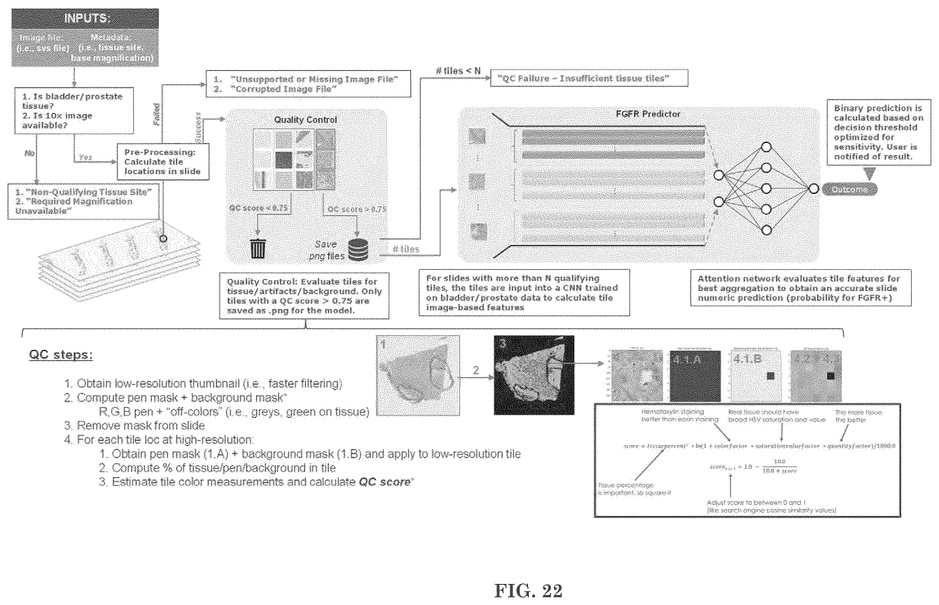

FIG. 22 depicts an example pipeline workflow. In particular, FIG. 22 shows several quality control steps that ensures that high quality images are analyzed by the neural network. Beginning at the top left of FIG. 22, the inputs to the pipeline workflow include the image file and associated metadata. A quality control step checks that the tissue is either bladder/prostate tissue and that a 10× zoom image is available. If either of these criteria are unmet, an indication that either criterion was not met can be provided. If the criteria are satisfied, then the image is pre-processed and tile locations in the slide are calculated. A second quality control step ensures that tile locations are successfully calculated in the image. If this step fails, an error can be provided. Otherwise, tiles are individually evaluated for presence of tissue, artifacts, and/or background.

Tiles are assigned a quality score according to the presence of tissue, artifacts, and/or background. For example, presence of tissue would increase the quality score whereas presence of artifacts and/or background would decrease the quality score. Further details of the steps for determining a quality score are shown in FIG. 22. Slides with more than N qualifying tiles are input into the neural network which is further connected to the attention network. Thus, the attention network outputs a binary prediction.

### ADDITIONAL EMBODIMENTS

Embodiments of the invention disclosed herein involve a pipeline workflow for predicting a subject tumor's genotype based on cancer tissue images captured from a sample obtained from the subject. Generally, the pipeline workflow involves 1) a step of pre-processing the image and 2) deployment of a trained neural network that analyzes individual tiles of an image and generates a prediction of the subject tumor's genotype. In various embodiments, the trained neural network includes a convolutional neural network that analyzes individual tiles and generates tile-level predictions, or, in alternative embodiments, tile-level features. In various embodiments, the trained neural network further includes an attention network that analyzes the tile-level predictions, or, alternatively, tile-level features, and aggregates the tile-level predictions to generate a prediction of the subject tumor's genotype, or, alternatively, aggregates tile-level features to generate an image-level feature vector used by connected a classification layer (or layers) to generate a prediction of the subject tumor's genotype. Altogether, the implementation of the pipeline workflow enables accurate prediction of subject genotypes that may be relevant drivers in certain cancers (e.g., bladder cancer or prostate cancer). Thus, this in silico process represents a rapid, low-cost procedure for determining whether a subject has a particular cancer-related genotype, which can further guide treatment options for the subject.

In various embodiments, subjects predicted to exhibit altered genotypes can undergo additional molecular testing to confirm the in silico results. In various embodiments, once confirmed through the molecular testing, appropriate medical treatment or medical advice can be provided to subjects based on the altered genotype status. In various embodiments, once confirmed, these subjects can be deemed eligible for enrollment in a clinical trial. In various embodiments, subjects predicted to exhibit wildtype genotypes are likely ineligible for the clinical trial and need not undergo further molecular testing, thereby saving resources (e.g., time and money). In various embodiments, the methods and/or non-transitory computer readable media described herein can operate as a Software as a Medical Device (SaMD) that is provided either on disk or via download or as a web-based software. In such embodiments, the methods described herein can operate independent of a clinical trial setting.

Disclosed herein is a method comprising: obtaining or having obtained an image of a tumor tissue slide from a subject; pre-processing the obtained image; applying a neural network model to analyze a plurality of tiles of the pre-processed image to generate a slide-level prediction; and determining a genotype of the subject using the slide-level prediction.

In various embodiments, pre-processing the obtained image comprises: performing a quality control process by removing tiles with excessive background and/or pen marks present in the image. In various embodiments, pre-processing the obtained image comprises one or more of: removing uninformative regions of one or more tiles of the image; performing an image stain augmentation of the obtained image. In various embodiments, performing the image stain augmentation comprises performing one or more of: changing stain intensity or stain contrast of one or more tiles of the obtained image, performing color jittering or color normalization of one or more tiles of the obtained image; and performing horizontal or vertical flips of one or more tiles of the obtained image. In various embodiments, pre-processing the obtained image is performed through an automated process.

In various embodiments, the neural network model comprises a convolutional neural network submodel and an attention network submodel. In various embodiments, the convolutional neural network submodel generates tile-level predictions for one or more tiles of the pre-processed image. In various embodiments, the attention network submodel receives the tile-level predictions of the convolutional neural network submodel and generates the image-level prediction. In various embodiments, the convolutional neural network submodel and the attention network submodel each comprise one or more layers of nodes. In various embodiments, the convolutional neural network submodel and the attention network submodel each comprise one or more learned parameters. In various embodiments, the one or more learned parameters of the attention network submodel guide aggregation of the tile-level predictions from the convolutional neural network submodel.

In various embodiments, the image-level prediction is a presence or absence of a genotype alteration. In various embodiments, the genotype alterations comprise a FGFR alteration. In various embodiments, the FGFR alteration comprises any of FGFR3 mutation, FGFR3 fusion, or a combination of a FGFR3 mutation and FGFR3 fusion. In various embodiments, the FGFR alteration is any of a p.R248C mutation, a p.G370C mutation, a p.S249C mutation, or a p.Y373C mutation. In various embodiments, the FGFR alteration is any of a FGFR3:TACC3V1 fusion, a FGFR3:TACC3V3 fusion, a FGFR3:BAIAP2L1 fusion, a FGFR2: BICC1 fusion, or a FGFR2:CASP7 fusion.

In various embodiments, the tumor tissue slide from the subject comprises bladder cancer. In various embodiments, the genotype alterations comprise single nucleotide polymorphisms (SNPs), copy number variations (CNVs), or gene fusions involving BRCA1 or BRCA2 genes. In various embodiments, the tumor tissue slide from the subject comprises prostate cancer. In various embodiments, the image is a hematoxylin and eosin (H&E) stained histopathology image. In various embodiments, the subject has cancer or is suspected of having cancer.

In various embodiments, methods disclosed herein further comprise: based on the determined genotype, determining whether to perform further molecular testing to confirm the determined genotype. In various embodiments, determining whether to perform further molecular testing comprises: responsive to the determined genotype indicating a genotype alteration, prioritizing the subject for undergoing further molecular testing. In various embodiments, determining whether to perform further molecular testing comprises: responsive to the determined genotype indicating a wildtype genotype, excluding the subject from undergoing further molecular testing.

In various embodiments, methods disclosed herein further comprise: determining whether to enroll the subject in a clinical trial according to at least the determined genotype. In various embodiments, determining whether to enroll the subject comprises: determining that the determined genotype comprises a genotype alteration; and determining that the subject is eligible for enrollment in the clinical trial based on at least the determination that the genotype comprises a genotype alteration. In various embodiments, determining that the subject is eligible for enrollment in the clinical trial is further based upon a molecular test that confirms that the subject exhibits a genotype that comprises the genotype alteration. In various embodiments, determining whether to enroll the subject comprises: determining that the determined genotype does not comprise a genotype alteration; and determining that the subject is ineligible for enrollment in the clinical trial based on at least the determination that the genotype does not comprise a genotype alteration. In various embodiments, based on the determination that the determined genotype does not comprise the genotype alteration, the subject does not undergo further molecular testing.

In various embodiments, methods disclosed herein further comprise: determining whether to administer a therapeutic according to at least the determined genotype. In various embodiments, the therapeutic is a FGFR kinase inhibitor. In various embodiments, the FGFR kinase inhibitor is Erdafitinib (BALVERSA). In various embodiments, the therapeutic is a PARP inhibitor. In various embodiments, the PARP inhibitor is Niraparib (ZEJULA). In various embodiments, the neural network model exhibits an auROC performance metric of at least 0.82 on bladder cancer images. In various embodiments, the neural network model exhibits a positive predictive value (PPV) performance metric of at least 0.22 at 100% recall on bladder cancer images in a test dataset with a 14% baseline prevalence for FGFR. In various embodiments, the neural network model exhibits an auROC performance metric of at least 0.71 on prostate cancer images. In various embodiments, the neural network model exhibits a positive predictive value (PPV) performance metric of at least 0.14 at 100% recall on prostate cancer images in a test dataset with a 11% baseline prevalence for PARP. In some examples, a neural network model consistent with the present disclosure exhibits an auROC performance metric of at least 0.78+/−0.03 for predicting MET genotypes in the context of non-small-cell lung cancer tissue images. In various embodiments, methods disclosed herein further comprise: reporting one or more tiles of the image that are most strongly associated with the genotype of the subject.

Additionally disclosed herein is a method comprising: obtaining or having obtained training data comprising training histopathology images; training a neural network model by analyzing a plurality of tiles of the training histopathology images, the neural network model configured to generate an image-level prediction informative for determining genotypes corresponding to the training histopathology images. In various embodiments, the neural network model comprises a convolutional neural network submodel and an attention network submodel. In various embodiments, the attention network submodel is trained using the training data comprising training histopathology images. In various embodiments, the convolutional neural network submodel and attention network submodel are separately trained. In various embodiments, the convolutional neural network submodel and attention network submodel are jointly trained using the training data.

In various embodiments, the training data further comprises reference ground truth labels indicating genotypes of training histopathology images. In various embodiments, the training histopathology images are obtained from a publicly available database. In various embodiments, the publicly available database is The Cancer Genome Atlas (TCGA) database. In various embodiments, the training histopathology images are obtained from a privately held database.

In various embodiments, the convolutional neural network submodel generates tile-level predictions for one or more tiles of the pre-processed image. In various embodiments, the attention network submodel receives the tile-level predictions of the neural network submodel and generates the image-level prediction. In various embodiments, the convolutional neural network submodel and the attention network submodel each comprise one or more layers of nodes. In various embodiments, the convolutional neural network submodel and the attention network submodel each comprise one or more learned parameters.

Additionally disclosed herein is a non-transitory computer readable medium comprising instructions that, when executed by a processor cause the processor to: obtain an image of a tumor tissue slide from a subject; pre-process the obtained image; apply a neural network model to analyze a plurality of tiles of the pre-processed image to generate a slide-level prediction; and determine a genotype of the subject using the slide-level prediction. In various embodiments, the instructions that cause the processor to pre-process the obtained image further comprise instructions that, when executed by the processor, cause the processor to: perform a quality control process by removing tiles with image background and/or pen marks that are present in the image. In various embodiments, the instructions that cause the processor to pre-process the obtained image further comprise instructions that, when executed by the processor, cause the processor to perform one or both of: remove uninformative regions of one or more tiles of the image; and perform an image stain augmentation of the obtained image. In various embodiments, the instructions that cause the processor to perform the image stain augmentation further comprise instructions that when executed by the processor, cause the processor to: change stain intensity or stain contrast of one or more tiles of the obtained image, perform color jittering or color normalization of one or more tiles of the obtained image; and perform horizontal or vertical flips of one or more tiles of the obtained image. In various embodiments, wherein the instructions that cause the processor to pre-process the obtained image enables the processor to perform the pre-processing through an automated process.

In various embodiments, the neural network model comprises a convolutional neural network submodel and an attention network submodel. In various embodiments, the convolutional neural network submodel generates tile-level predictions for one or more tiles of the pre-processed image. In various embodiments, the attention network submodel receives the tile-level predictions of the convolutional neural network submodel and generates the image-level prediction. In various embodiments, the convolutional neural network submodel and the attention network submodel each comprise one or more layers of nodes. In various embodiments, the convolutional neural network submodel and the attention network submodel each comprise one or more learned parameters. In various embodiments, the one or more learned parameters of the attention network submodel guide aggregation of the tile-level predictions from the convolutional neural network submodel.

In various embodiments, the image-level prediction is a presence or absence of a genotype alteration. In various embodiments, the genotype alterations comprise a FGFR alteration. In various embodiments, the FGFR alteration comprises any of FGFR3 mutation, FGFR3 fusion, or a combination of a FGFR3 mutation and FGFR3 fusion. In various embodiments, the FGFR alteration is any of a p.R248C mutation, a p.G370C mutation, a p.S249C mutation, or a p.Y373C mutation. In various embodiments, the FGFR alteration is any of a FGFR3:TACC3V1 fusion, a FGFR3:TACC3V3 fusion, a FGFR3:BAIAP2L1 fusion, a FGFR2: BICC1 fusion, or a FGFR2:CASP7 fusion.

In various embodiments, the tumor tissue slide from the subject comprises bladder cancer. In various embodiments, the genotype alterations comprise single nucleotide polymorphisms (SNPs), copy number variations (CNVs), or gene fusions involving BRCA1 or BRCA2 genes. In various embodiments, the tumor tissue slide from the subject comprises prostate cancer. In various embodiments, the image is a hematoxylin and eosin (H&E) stained histopathology image. In various embodiments, the subject has cancer or is suspected of having cancer.

In various embodiments, the non-transitory computer readable medium further comprises instructions that, when executed by the processor, cause the processor to: based on the determined genotype, determine whether to perform further molecular testing to confirm the determined genotype. In various embodiments, the instructions that cause the processor to determine whether to perform further molecular testing further comprises instructions that, when executed by the processor, cause the processor to: responsive to the determined genotype indicating a genotype alteration, prioritize the subject for undergoing further molecular testing. In various embodiments, the instructions that cause the processor to determine whether to perform further molecular testing further comprises instructions that, when executed by the processor, cause the processor to: responsive to the determined genotype indicating a wildtype genotype, exclude the subject from undergoing further molecular testing.

In various embodiments, the non-transitory computer readable medium further comprises instructions that, when executed by the processor, cause the processor to: determine whether to enroll the subject in a clinical trial according to at least the determined genotype. In various embodiments, the instructions that cause the processor to determine whether to enroll the subject further comprise instructions that, when executed by the processor, cause the processor to: determine that the determined genotype comprises a genotype alteration; and determine that the subject is eligible for enrollment in the clinical trial based on at least the determination that the genotype comprises a genotype alteration. In various embodiments, the determination that the subject is eligible for enrollment in the clinical trial is further based upon a molecular test that confirms that the subject exhibits a genotype that comprises the genotype alteration. In various embodiments, the instructions that cause the processor to determine whether to enroll the subject further comprise instructions that, when executed by the processor, cause the processor to: determine that the determined genotype does not comprise a genotype alteration; and determine that the subject is ineligible for enrollment in the clinical trial based on at least the determination that the genotype does not comprise a genotype alteration. In various embodiments, based on the determination that the determined genotype does not comprise the genotype alteration, the subject does not undergo further molecular testing.

In various embodiments, the non-transitory computer readable medium further comprises instructions that, when executed by the processor, cause the processor to: determine whether to administer a therapeutic according to at least the determined genotype. In various embodiments, the therapeutic is a FGFR kinase inhibitor. In various embodiments, the FGFR kinase inhibitor is Erdafitinib (BALVERSA). In various embodiments, the therapeutic is a PARP inhibitor. In various embodiments, the PARP inhibitor is Niraparib (ZEJULA).

In various embodiments, the neural network model exhibits an auROC performance metric of at least 0.82 on bladder cancer models. In various embodiments, the neural network model exhibits a positive predictive value (PPV) performance metric of at least 0.22 at 100% recall on bladder cancer models in a test dataset with a 14% baseline prevalence for FGFR. In various embodiments, the neural network model exhibits an auROC performance metric of at least 0.71 on prostate cancer models. In various embodiments, the neural network model exhibits a positive predictive value (PPV) performance metric of at least 0.14 at 100% recall on prostate cancer images in a test dataset with a 11% baseline prevalence for PARP. In some examples, a neural network model consistent with the present disclosure exhibits an auROC performance metric of at least 0.78+/−0.03 for predicting MET genotypes in the context of non-small-cell lung cancer tissue images. In various embodiments, the non-transitory computer readable medium further comprises instructions that, when executed by the processor, cause the processor to: report one or more tiles of the image that are most strongly associated with the genotype of the subject.

Additionally disclosed herein is a non-transitory computer readable medium comprising instructions that, when executed by the processor, cause the processor to: obtain training data comprising training histopathology images; train a neural network model by analyzing a plurality of tiles of the training histopathology images, the neural network model configured to generate an image-level prediction informative for determining genotypes corresponding to the training histopathology images. In various embodiments, the neural network model comprises a convolutional neural network submodel and an attention network submodel. In various embodiments, the attention network submodel is trained using the training data comprising training histopathology images. In various embodiments, the convolutional neural network submodel and attention network submodel are separately trained. In various embodiments, the convolutional neural network submodel and attention network submodel are jointly trained using the training data.

In various embodiments, the training data further comprises reference ground truth labels indicating genotypes of training histopathology images. In various embodiments, the training histopathology images are obtained from a publicly available database. In various embodiments, the publicly available database is The Cancer Genome Atlas (TCGA) database. In various embodiments, the training histopathology images are obtained from a privately held database.

In various embodiments, the convolutional neural network submodel generates tile-level predictions for one or more tiles of the pre-processed image. In various embodiments, the attention network submodel receives the tile-level predictions of the neural network submodel and generates the image-level prediction. In various embodiments, the convolutional neural network submodel and the attention network submodel each comprise one or more layers of nodes. In various embodiments, the convolutional neural network submodel and the attention network submodel each comprise one or more learned parameters.

Embodiment 1: A method comprising: obtaining or having obtained an image of a tumor tissue slide from a subject; pre-processing the obtained image; applying a neural network model to analyze a plurality of tiles of the pre-processed image to generate a slide-level prediction, or, optionally, slide-level features; and determining a genotype of the subject using the slide-level prediction or the slide-level features.

Embodiment 2: The method of embodiment 1, wherein pre-processing the obtained image comprises: performing a quality control process by removing tiles with image background and/or pen marks that are present in the image.

Embodiment 3: The method of embodiment 1, wherein pre-processing the obtained image comprises one or more of: removing uninformative regions of one or more tiles of the image; performing an image stain augmentation of the obtained image.

Embodiment 4: The method of embodiment 3, wherein performing the image stain augmentation comprises performing one or more of: changing stain intensity or stain contrast of one or more tiles of the obtained image, performing color jittering or color normalization of one or more tiles of the obtained image; and performing horizontal or vertical flips of one or more tiles of the obtained image.

Embodiment 5: The method of any one of embodiments 1-4, wherein pre-processing the obtained image is performed through an automated process.

Embodiment 6: The method of embodiment 1, wherein the neural network model comprises a convolutional neural network submodel and an attention network submodel.

Embodiment 7: The method of embodiment 6, wherein the convolutional neural network submodel generates tile-level predictions for one or more tiles of the pre-processed image.

Embodiment 8: The method of embodiment 6 or 7, wherein the attention network submodel receives the tile-level predictions of the convolutional neural network submodel and generates the image-level prediction.

Embodiment 9: The method of any one of embodiments 6-8, wherein the convolutional neural network submodel and the attention network submodel each comprise one or more layers of nodes.

Embodiment 10: The method of any one of embodiments 6-9, wherein the convolutional neural network submodel and the attention network submodel each comprise one or more learned parameters.

Embodiment 11: The method of embodiment 10, wherein the one or more learned parameters of the attention network submodel guide aggregation of the tile-level predictions from the convolutional neural network submodel.

Embodiment 12: The method of any one of embodiments 1-11, wherein the image-level prediction is a presence or absence of a genotype alteration.

Embodiment 13: The method of embodiment 12, wherein the genotype alterations comprise a FGFR alteration.

Embodiment 14: The method of embodiment 13, wherein the FGFR alteration comprises any of FGFR3 mutation, FGFR3 fusion, or a combination of a FGFR3 mutation and FGFR3 fusion.

Embodiment 15: The method of embodiment 13, wherein the FGFR alteration is any of a p.R248C mutation, a p.G370C mutation, a p.S249C mutation, or a p.Y373C mutation.

Embodiment 16: The method of embodiment 13, wherein the FGFR alteration is any of a FGFR3:TACC3V1 fusion, a FGFR3:TACC3V3 fusion, a FGFR3:BAIAP2L1 fusion, a FGFR2: BICC1 fusion, or a FGFR2:CASP7 fusion.

Embodiment 17: The method of any one of embodiments 13-16, wherein the tumor tissue slide from the subject comprises bladder cancer.

Embodiment 18: The method of embodiment 12, wherein the genotype alterations comprise single nucleotide polymorphisms (SNPs), copy number variations (CNVs), or gene fusions involving BRCA1 or BRCA2 genes.

Embodiment 19: The method of embodiment 18, wherein the tumor tissue slide from the subject comprises prostate cancer.

Embodiment 20: The method of any of embodiments 1-19, wherein the image is a hematoxylin and eosin (H&E) stained histopathology image.

Embodiment 21: The method of any one of embodiments 1-20, wherein the subject has cancer or is suspected of having cancer.

Embodiment 22: The method of any one of embodiments 1-21, further comprising:

based on the determined genotype, determining whether to perform further molecular testing to confirm the determined genotype.

Embodiment 23: The method of embodiment 22, wherein determining whether to perform further molecular testing comprises: responsive to the determined genotype indicating a genotype alteration, prioritizing the subject for undergoing further molecular testing.

Embodiment 24: The method of embodiment 22, wherein determining whether to perform further molecular testing comprises: responsive to the determined genotype indicating a wildtype genotype, excluding the subject from undergoing further molecular testing.

Embodiment 25: The method of any one of embodiments 1-21, further comprising: determining whether to enroll the subject in a clinical trial according to at least the determined genotype.

Embodiment 26: The method of embodiment 22, wherein determining whether to enroll the subject comprises: determining that the determined genotype comprises a genotype alteration; and determining that the subject is eligible for enrollment in the clinical trial based on at least the determination that the genotype comprises a genotype alteration.

Embodiment 27: The method of embodiment 26, wherein determining that the subject is eligible for enrollment in the clinical trial is further based upon a molecular test that confirms that the subject exhibits a genotype that comprises the genotype alteration.

Embodiment 20: The method of embodiment 22, wherein determining whether to enroll the subject comprises: determining that the determined genotype does not comprise a genotype alteration; and determining that the subject is ineligible for enrollment in the clinical trial based on at least the determination that the genotype does not comprise a genotype alteration.

Embodiment 29: The method of embodiment 28, wherein based on the determination that the determined genotype does not comprise the genotype alteration, the subject does not undergo further molecular testing.

Embodiment 30: The method of any one of embodiments 1-22, further comprising: determining whether to administer a therapeutic according to at least the determined genotype.

Embodiment 31: The method of embodiment 30, wherein the therapeutic is a FGFR kinase inhibitor.

Embodiment 32: The method of embodiment 31, wherein the FGFR kinase inhibitor is Erdafitinib (BALVERSA).

Embodiment 33: The method of embodiment 30, wherein the therapeutic is a PARP inhibitor.

Embodiment 34: The method of embodiment 33, wherein the PARP inhibitor is Niraparib (ZEJULA).

Embodiment 35: The method of any one of embodiments 1-5 and 12-34, wherein the neural network model exhibits an auROC performance metric of at least 0.82 on bladder cancer models.

Embodiment 36: The method of any one of embodiments 1-5 and 12-34, wherein the neural network model exhibits a positive predictive value (PPV) performance metric of at least 0.22 at 100% recall on bladder cancer images in a test dataset with a 14% baseline prevalence for FGFR.

Embodiment 37: The method of any one of embodiments 6-34, wherein the neural network model exhibits an auROC performance metric of at least 0.71 on prostate cancer images.

Embodiment 37.5: The method of any one of embodiments 6-34, wherein the neural network model exhibits an auROC performance metric of at least 0.78+/−0.03 for predicting MET genotypes on non-small-cell lung cancer images.

Embodiment 38: The method of any one of embodiments 6-34, wherein the neural network model exhibits a positive predictive value (PPV) performance metric of at least 0.14 at 100% recall on prostate cancer models in a test dataset with a 11% baseline prevalence for PARP.

Embodiment 39: The method of any one of embodiments 1-38, further comprising: reporting one or more tiles of the image that are most strongly associated with the genotype of the subject.

Embodiment 40: A method comprising: obtaining or having obtained an image of a tumor tissue slide from a subject; pre-processing the obtained image; applying a neural network model to analyze a plurality of tiles of the pre-processed image to generate a slide-level prediction; determining a genotype of the subject using the slide-level prediction.

Embodiment 41: The method of embodiment 40, wherein pre-processing the obtained image comprises: performing a quality control process by removing tiles with image background and/or pen marks that are present in the image.

Embodiment 42: The method of embodiment 40, wherein pre-processing the obtained image comprises one or more of: removing uninformative regions of one or more tiles of the image; performing an image stain augmentation of the obtained image.

Embodiment 43: The method of embodiment 42, wherein performing the image stain augmentation comprises performing one or more of: changing stain intensity or stain contrast of one or more tiles of the obtained image, performing color jittering or color normalization of one or more tiles of the obtained image; and performing horizontal or vertical flips of one or more tiles of the obtained image.

Embodiment 44: The method of any one of embodiments 40-43, wherein pre-processing the obtained image is performed through an automated process.

Embodiment 45: The method of embodiment 40, wherein the neural network model comprises a convolutional neural network submodel and an attention network submodel.

Embodiment 46: The method of embodiment 45, wherein the convolutional neural network submodel generates tile-level predictions for one or more tiles of the pre-processed image.

Embodiment 47: The method of embodiment 45 or 46, wherein the attention network submodel receives the tile-level predictions of the convolutional neural network submodel and generates the image-level prediction.

Embodiment 48: The method of any one of embodiments 45-47, wherein the convolutional neural network submodel and the attention network submodel each comprise one or more layers of nodes.

Embodiment 49: The method of any one of embodiments 45-48, wherein the convolutional neural network submodel and the attention network submodel each comprise one or more learned parameters.

Embodiment 50: The method of embodiment 49, wherein the one or more learned parameters of the attention network submodel guide aggregation of the tile-level predictions from the convolutional neural network submodel.

Embodiment 51: The method of any one of embodiments 40-50, wherein the image-level prediction is a presence or absence of a genotype alteration.

Embodiment 52: The method of embodiment 51, wherein the genotype alterations comprise a FGFR alteration.

Embodiment 53: The method of embodiment 52, wherein the FGFR alteration comprises any of FGFR3 mutation, FGFR3 fusion, or a combination of a FGFR3 mutation and FGFR3 fusion.

Embodiment 54: The method of embodiment 52, wherein the FGFR alteration is any of a p.R248C mutation, a p.G370C mutation, a p.S249C mutation, or a p.Y373C mutation.

Embodiment 55: The method of embodiment 52, wherein the FGFR alteration is any of a FGFR3:TACC3V1 fusion, a FGFR3:TACC3V3 fusion, a FGFR3:BAIAP2L1 fusion, a FGFR2: BICC1 fusion, or a FGFR2:CASP7 fusion.

Embodiment 56: The method of any one of embodiments 52-55, wherein the tumor tissue slide from the subject comprises bladder cancer.

Embodiment 57: The method of embodiment 51, wherein the genotype alterations comprise single nucleotide polymorphisms (SNPs), copy number variations (CNVs), or gene fusions involving BRCA1 or BRCA2 genes.

Embodiment 58: The method of embodiment 57, wherein the tumor tissue slide from the subject comprises prostate cancer.

Embodiment 59: The method of any one of embodiments 40-58, wherein the image is a hematoxylin and eosin (H&E) stained histopathology image.

Embodiment 60: The method of any one of embodiments 40-59, wherein the subject has cancer or is suspected of having cancer.

Embodiment 61: The method of any one of embodiments 40-60, further comprising: based on the determined genotype, determining whether to perform further molecular testing to confirm the determined genotype.

Embodiment 62: The method of embodiment 61, wherein determining whether to perform further molecular testing comprises: responsive to the determined genotype indicating a genotype alteration, prioritizing the subject for undergoing further molecular testing.

Embodiment 63: The method of embodiment 62, wherein determining whether to perform further molecular testing comprises: responsive to the determined genotype indicating a wildtype genotype, excluding the subject from undergoing further molecular testing.

Embodiment 64: The method of any one of embodiments 40-61, further comprising: determining whether to enroll the subject in a clinical trial according to at least the determined genotype.

Embodiment 65: The method of embodiment 61, wherein determining whether to enroll the subject comprises: determining that the determined genotype comprises a genotype alteration; and determining that the subject is eligible for enrollment in the clinical trial based on at least the determination that the genotype comprises a genotype alteration.

Embodiment 66: The method of embodiment 65, wherein determining that the subject is eligible for enrollment in the clinical trial is further based upon a molecular test that confirms that the subject exhibits a genotype that comprises the genotype alteration.

Embodiment 67: The method of embodiment 66, wherein determining whether to enroll the subject comprises: determining that the determined genotype does not comprise a genotype alteration; and determining that the subject is ineligible for enrollment in the clinical trial based on at least the determination that the genotype does not comprise a genotype alteration.

Embodiment 68: The method of embodiment 67, wherein based on the determination that the determined genotype does not comprise the genotype alteration, the subject does not undergo further molecular testing.

Embodiment 69: The method of any one of embodiments 40-61, further comprising:
determining whether to administer a therapeutic according to at least the determined genotype.

Embodiment 70: The method of embodiment 69, wherein the therapeutic is a FGFR kinase inhibitor.

Embodiment 71: The method of embodiment 70, wherein the FGFR kinase inhibitor is Erdafitinib (BALVERSA).

Embodiment 72: The method of embodiment 69, wherein the therapeutic is a PARP inhibitor.

Embodiment 73: The method of embodiment 69, wherein the PARP inhibitor is Niraparib (ZEJULA).

Embodiment 74: The method of any one of embodiments 40-44 and 51-63, wherein the neural network model exhibits an auROC performance metric of at least 0.82 on bladder cancer models.

Embodiment 75: The method of any one of embodiments 40-44 and 51-63 wherein the neural network model exhibits a positive predictive value (PPV) performance metric of at least 0.22 at 100% recall on bladder cancer models in a test dataset with a 14% baseline prevalence for FGFR.

Embodiment 76: The method of any one of embodiments 45-73, wherein the neural network model exhibits an auROC performance metric of at least 0.71 on prostate cancer models.

Embodiment 77: The method of any one of embodiments 45-73, wherein the neural network model exhibits a positive predictive value (PPV) performance metric of at least 0.14 at 100% recall on prostate cancer models in a test dataset with a 11% baseline prevalence for PARP.

Embodiment 78: The method of any one of embodiments 40-77, further comprising: reporting one or more tiles of the image that are most strongly associated with the genotype of the subject.

Embodiment 79: A method comprising: obtaining or having obtained training data comprising training histopathology images; training a neural network model by analyzing a plurality of tiles of the training histopathology images, the neural network model configured to generate an image-level prediction informative for determining genotypes corresponding to the training histopathology images.

Embodiment 80: The method of embodiment 79, wherein the neural network model comprises a convolutional neural network submodel and an attention network submodel.

Embodiment 81: The method of embodiment 80, wherein the attention network submodel is trained using the training data comprising training histopathology images.

Embodiment 82: The method of embodiment 81, wherein the convolutional neural network submodel and attention network submodel are separately trained.

Embodiment 83: The method of embodiment 81, wherein the convolutional neural network submodel and attention network submodel are jointly trained using the training data.

Embodiment 84: The method of any one of embodiments 79-83, wherein the training data further comprises reference ground truth labels indicating genotypes of training histopathology images.

Embodiment 85: The method of any one of embodiments 79-84, wherein the training histopathology images are obtained from a publicly available database.

Embodiment 86: The method of embodiment 85, wherein the publicly available database is The Cancer Genome Atlas (TCGA) database.

Embodiment 87: The method of any one of embodiments 79-84, wherein the training histopathology images are obtained from a privately held database.

Embodiment 88: The method of any one of embodiments 80-87, wherein the convolutional neural network submodel generates tile-level predictions for one or more tiles of the pre-processed image.

Embodiment 89: The method of any one of embodiments 80-88, wherein the attention network submodel receives the tile-level predictions of the neural network submodel and generates the image-level prediction.

Embodiment 90: The method of any one of embodiments 80-89, wherein the convolutional neural network submodel and the attention network submodel each comprise one or more layers of nodes.

Embodiment 91: The method of any one of embodiments 80-90, wherein the convolutional neural network submodel and the attention network submodel each comprise one or more learned parameters.

Embodiment 92: A non-transitory computer readable medium comprising instructions that, when executed by a processor, cause the processor to perform operations comprising steps in the method of any one of embodiments 1-91.

While the present disclosure has been particularly described with respect to the illustrated embodiments, it will be appreciated that various alterations, modifications, and adaptations may be made based on the disclosure and are intended to be within the scope of the disclosure. While the disclosure has been described in connection with what are presently considered to be the most practical and preferred embodiments, it is to be understood that the present disclosure is not limited to the disclosed embodiments but, on the contrary, is intended to cover various modifications and equivalent arrangements included within the scope of the underlying principles of the invention as described by the various embodiments reference above and below.

1. A method of, via a deep learning pipeline, generating a deep learning network configured to execute on one or more computers to use histopathology image data from a histopathology image corresponding to member of a cohort of interest to generate a prediction relevant to likelihood of therapeutic response to a new treatment in the member the cohort of interest, the cohort of interest comprising candidates for receiving the new treatment in a clinical trial, the method comprising:
training, in succession, a plurality of respective deep learning networks from a first deep learning network to a last deep learning network using respective histopathology image datasets having respective degrees of relevance to the cohort of interest; and
transferring, in succession, learned parameters of one deep learning network of the plurality of respective deep learning networks to another deep learning network of the plurality of respective deep learning networks after training the one deep learning network with a one of the respective histopathology image datasets and before training the another deep learning network with another histopathology image dataset of the respective histopathology image datasets.

2. The method of claim 1 wherein the respective degrees of relevance to the cohort of interest increase from a first respective histopathology image dataset to a last respective histopathology image dataset used in training the respective deep learning networks.

3. The method of claim 2 wherein the first histopathology image dataset is significantly larger than the last histopathology image dataset.

4. The method of claim 1 wherein a first histopathology image dataset of the respective histopathology image datasets comprises unlabeled histopathology image data.

5. The method of claim 4 wherein the first deep learning network comprises a feature extraction network and a contrastive learning module.

6. The method of claim 5 wherein the first deep learning network further comprises a projection network configured to receive feature vectors from the feature extraction network and to provide feature vectors to the contrastive learning module.

7. The method of claim 1 wherein each deep learning network of the plurality of respective deep learning networks from a second deep learning network to the last deep learning network comprises a feature extraction network and a classification network, and further wherein the second deep learning network to the last deep learning network is trained using supervised learning.

8. The method of claim 7 wherein the each deep learning network from the second deep learning network to the last deep learning network further comprises an attention network.

9. The method of claim 8 further comprising, for each of the second deep learning network to the last deep learning network, combining output the feature extraction layers and the attention network to provide a combined output to a classification network.

10. (canceled)

11. The method of claim 10 wherein, for each feature vector obtained from data corresponding to a particular histopathology image, the each feature vector is multiplied by a corresponding attention value and the results are combined to produce a summarized feature vector summarizing all feature vectors obtained from the data corresponding to the particular histopathology image.

12. The method of claim 10 wherein the summarized feature vector is obtained by averaging the results of multiplying each feature vector by the corresponding attention value.

13. The method of claim 12 wherein the summarized feature vector is submitted to a classification network.

14. (canceled)

15. The method of claim 1, wherein the prediction relevant to likelihood of therapeutic responses comprises a prediction regarding a presence or absence of a genotype alteration corresponding to tumor tissue in the histopathology image.

16. The method of claim 15, wherein the genotype alteration comprises a fibroblast growth factor receptor (FGFR) alteration.

17. The method of claim 16, wherein the FGFR alteration comprises any of FGFR3 mutation, FGFR3 fusion, or a combination of a FGFR3 mutation and FGFR3 fusion.

18. The method of claim 16, wherein the FGFR alteration is any of a p.R248C mutation, a p.G370C mutation, a p.S249C mutation, or a p.Y373C mutation.

19. The method of claim 16, wherein the FGFR alteration is any of a FGFR3:TACC3V1 fusion, a FGFR3:TACC3V3 fusion, a FGFR3:BAIAP2L1 fusion, a FGFR2: BICC1 fusion, or a FGFR2:CASP7 fusion.

20. The method of claim 15, wherein the tumor tissue comprises bladder cancer.

21. The method of claim 15, wherein the genotype alteration comprises one or more of a single nucleotide polymorphism (SNP), copy number variation (CNV), gene fusion, or a DNA repair deficiency (DRD) involving BRCA1, BRCA2, BRIP1, CDK12, CHEK2, FANCA, PALB2, RAD51B, RAD54L, RAD21, or SPOP.

22. The method of claim 15, wherein the tumor tissue comprises prostate cancer.

23-24. (canceled)

25. The method of claim 15, further comprising: based on the determined genotype, determining whether to perform further molecular testing to confirm the determined genotype.

26. The method of claim 25, wherein determining whether to perform further molecular testing comprises: responsive to the determined genotype indicating a genotype alteration, prioritizing the member of the cohort of interest for undergoing further molecular testing.

27. The method of claim 26, wherein determining whether to perform further molecular testing comprises: responsive to the determined genotype indicating a wildtype genotype, excluding the member of the cohort of interest from undergoing further molecular testing.

28. The method of claim 15, further comprising: determining whether to enroll the member of the cohort of interest in a clinical trial according to at least the determined genotype.

29. The method of claim 28, wherein determining whether to enroll the member of the cohort of interest comprises:
determining that the determined genotype comprises a genotype alteration; and
determining that the member of the cohort of interest is eligible for enrollment in the clinical trial based on at least the determination that the genotype comprises a genotype alteration.

30. The method of claim 29, wherein determining that the member of the cohort of interest is eligible for enrollment in the clinical trial is further based upon a molecular test that confirms that the member of the cohort of interest exhibits a genotype that comprises the genotype alteration.

31-32. (canceled)

33. The method of claim 15, further comprising: determining whether to administer a therapeutic according to at least the determined genotype.

34. The method of claim 33, wherein the therapeutic is a FGFR kinase inhibitor.

35. The method of claim 34, wherein the FGFR kinase inhibitor is erdafitinib.

36. The method of claim 33, wherein the therapeutic is a PARP inhibitor.

37. The method of claim 36, wherein the PARP inhibitor is niraparib.

38. The method of claim 33, wherein the therapeutic is a monoclonal antibody.

39. The method of claim 38, wherein the monoclonal antibody is amivantamab.

40-45. (canceled)

46. A computer program product stored in a non-transitory computer readable medium comprising instructions configured to execute a method according to claim 1 using one or more computer processors.

47. (canceled)

48. A system comprising:
a deep learning pipeline comprising one or more computers coupled to a non-transitory computer readable medium storing instructions that are executable by one or more processors of the one or more computers for training, in succession, a plurality of respective deep learning networks using respective histopathology image datasets, each respective histopathology image dataset having a respective degree of relevance to a cohort of interest, wherein training comprises:
training a first deep learning network of the plurality of deep learning networks with one histopathology image dataset of the respective histopathology image datasets;
transferring, in succession, a plurality of learned parameters of the first deep learning network to a second deep learning network of the plurality of respective deep learning networks; and
training the second deep learning network with another histopathology image dataset of the respective histopathology image datasets.

49. The method of claim 15, wherein the genotype alteration comprises an alteration in the MET gene.

50. The method of claim 49, wherein the tumor tissue comprises lung cancer.
